(19) United States
(12) Patent Application Publication
(10) Pub. No.: US 2016/0120492 A1
(43) Pub. Date: May 5, 2016

(54) FILLING MATERIAL AND METHOD OF CONFIRMING INJECTION STATE OF FILLING MATERIAL

(71) Applicant: TERUMO KABUSHIKI KAISHA, Tokyo (JP)

(72) Inventors: Yasuyuki Honma, Ashigarakami-gun (JP); Yasushi Kinoshita, Fujinomiya (JP); Tomomi Kitagawa, Besancon (FR)

(73) Assignee: TERUMO KABUSHIKI KAISHA, Tokyo (JP)

(21) Appl. No.: 14/991,197

(22) Filed: Jan. 8, 2016

Related U.S. Application Data
(63) Continuation of application No. PCT/JP2013/068832, filed on Jul. 10, 2013.

(51) Int. Cl. A61B 6/00; A61B 17/70; A61L 27/04; A61L 27/50; A61K 49/04

(52) U.S. Cl. CPC A61B 6/481; A61L 27/50; A61K 49/0414; A61L 27/047; A61B 17/7068; A61B 17/7097; A61B 17/7094; A61L 2430/38; A61L 2300/40

(57) ABSTRACT

A filling material which enables a user to visually confirm the state of the filling material while it is injected into a flexible container based on an X-ray image, and a method of confirming the injection state of the filling material. The filling material configured to be injected into the flexible container includes a first contrast substance that is flowable, and a second contrast substance that is mixed into the first contrast substance and is insoluble in the first contrast substance. The second contrast substance possesses higher contrast X-ray contrast properties than the X-ray contrast properties of the first contrast substance.

### CROSS-REFERENCES TO RELATED APPLICATIONS

This application is a continuation of International Application No. PCT/JP2013/068832 filed on Jul. 10, 2013, the entire content of which is incorporated herein by reference.

### TECHNICAL FIELD

The present invention generally relates to a filling material to be injected into a flexible container and a method of confirming an injection state of a filling material when the filling material is introduced into the inside of a container.

### BACKGROUND DISCUSSION

In the medical field, various medical practices are known in which a flexible container such as a balloon and the like is introduced into a living body and is expanded by injecting a filling material into the container. The expansion of the flexible container in this manner forms a cavity inside living body organs or broadens a gap between the adjacent living body organs. Alternatively, for example as described in Japanese Patent Application Publication No. 2011-521746, the container is caused to indwell to maintain the gap between the adjacent living body organs.

In the medical practices in which the container and the filling material are used as described above, injection work is carried out to optimize the injection amount of the filling material based on the intended use of the container. If the container is over expanded by excessively supplying the filling material into the container, the container may be damaged or the container may apply a heavy force to the living body organs around the container. On the other hand, if the container is under expanded because an insufficient amount of the filling material is injected into the container, the intended therapeutic effect may not be sufficiently obtained.

### SUMMARY

As a method of preventing the above-described problem from occurring, a user can confirm a state of the container while the filling material is being injected, for example, by using an X-ray imaging device after adding a material possessing X-ray contrast properties to the filling material. However, if the X-ray contrast properties are merely provided for the filling material, a uniform grayscale image of the whole filling material is projected onto the X-ray image. Consequently, the flowing state of the filling material cannot be visually confirmed. It is thus difficult to visually determine based on the X-ray image whether the container is fully filled with the filling material or whether the filling material is still being continuously injected into the container.

The filling material disclosed here addresses the problems discussed above by enabling a user to visually confirm the filling material state based on an X-ray image while the filling material is being injected into a flexible container, and to provide a method of confirming an injection state of the filling material.

The filling material disclosed here includes various aspects such as those summarized below.

(1) There is provided a filling material to be injected into an expandable and flexible container which is to be introduced into a living body. The filling material includes a first contrast substance that includes X-ray contrast properties and fluidity, and a second contrast substance that includes insolubility in the first contrast substance and the X-ray contrast properties which are higher than those of the first contrast substance, and that is mixed in the first contrast substance.

(2) In the filling material according to (1) described above, a brightness difference between the second contrast substance and the first contrast substance is five or more on an X-ray image.

(3) In the filling material according to (1) or (2) described above, the second contrast substance is scattered in a particle shape in the first contrast substance.

(4) In the filling material according to (3) described above, a particle size of the second contrast substance is equal to or larger than 0.007 mm, and is smaller than 2.0 mm.

(5) In the filling material according to any one of (1) to (4) described above, the first contrast substance is configured to be cured with the lapse of time.

(6) In the filling material according to any one of (1) to (5) described above, the second contrast substance includes a metal material.

(7) In the filling material according to any one of (1) to (6) described above, the container is a medical device which is caused to indwell between adjacent spinous processes inside a living body in a state of having the injected filling material so as to maintain a gap between spinous processes. The filling material is used so as to expand the container and so as to maintain an expansion state of the container.

(8) There is provided a method of confirming an injection state of a filling material, when the filling material which has a first contrast substance including X-ray contrast properties and fluidity and a second contrast substance including insolubility in the first contrast substance and the X-ray contrast properties which are higher than those of the first contrast substance and being mixed in the first contrast substance is injected into a flexible container. The method includes observing the second contrast substance on an X-ray image, and confirming a state where the filling material is injected into the container, based on an observation result.

According to the filling material aspect in (1) described above, the X-ray contrast properties of the second contrast substance which is mixed in the first contrast substance including the fluidity are higher than the X-ray contrast properties of the first contrast substance. Therefore, a user can visually and easily identify the first contrast substance and the second contrast substance on the X-ray image. Then, a user can recognize a flowing state of the whole filling material by observing the movement of the second contrast substance when the user carries out injection work of the filling material. Therefore, the user can easily confirm a state where the filling material is injected into the container, based on the user's observation result.

According to the filling material aspect in (2) described above, the brightness difference between the first contrast substance and the second contrast substance is set to five or more on the X-ray image. Therefore, a user can more reliably identify the first contrast substance and the second contrast substance, and can smoothly carry out work for confirming an injection state of the filling material.

According to the filling material aspect in (3) described above, the second contrast substance is scattered in a particle shape in the first contrast substance. Therefore, a user can more easily observe each movement of the second contrast substance in the respective substances.

According to the filling material aspect in (4) described above, the particle size of the second contrast substance is formed so as to be equal to or larger than 0.007 mm, and so as to be smaller than 2.0 mm. Therefore, the second contrast substance can be displayed using a visible size when a user refers to a captured X-ray image, thereby enabling the user to more conveniently and quickly carry out work for confirming an injection state of the filling material.

According to the filling material aspect in (5) described above, the first contrast substance is configured to be cured with the lapse of time. Therefore, a user can preferably employ the filling material for the user's manual skills when causing the container to indwell in a living body so as to maintain and expand a gap between living body organs.

According to the filling material aspect in (6) described above, the second contrast substance includes the metal material. Therefore, a user can conveniently adjust the X-ray contrast properties so that the X-ray contrast properties of the second contrast substance become higher than the X-ray contrast properties of the first contrast substance. In addition, the user can adjust the X-ray contrast properties by adding the existing metal material having the X-ray contrast properties to the second contrast substance. Therefore, it is possible to suppress an increase in manufacturing cost for the filling material.

According to the filling material aspect in (7) described above, the container having the injected filling material can preferably maintain a gap between adjacent spinous processes. In addition, a user can confirm an expansion state of the container by observing the movement of the second contrast substance, when carrying out work for injecting the filling material into the container. Therefore, the user can more conveniently and quickly use the user's manual skills in order to maintain a gap between spinous processes.

According to the method in (8) described above, a user can recognize a flowing state of the whole filling material by observing the movement of the second contrast substance which is mixed in the first contrast substance, when carrying out work for injecting the filling material into the flexible container introduced into a living body. Therefore, the user can easily confirm a state where the filling material is injected into the container.

### BRIEF DESCRIPTION OF THE DRAWINGS

FIGS. 1A and 1B are views illustrating a living body for the manually operated device and filling material according to an embodiment of this application. FIG. 1A is a view schematically illustrating a back of the living body, and FIG. 1B is a view schematically illustrating a spinous process and a peripheral site in the living body.

FIGS. 2A, 2B and 2C are views illustrating a manually operated medical device that uses the filling material according to the embodiment. FIG. 2A is a side view of a puncture member included in the medical device, FIG. 2B is a side view of a guide member used together with the puncture member, and FIG. 2C is a side view of the medical device with the puncture member and the guide member assembled together.

FIG. 3 is a view schematically illustrating the needle portion of the puncture member puncturing the living body.

FIG. 4 is a view schematically illustrating a flexible container being introduced into the living body by using the guide member.

FIG. 5 is a view schematically illustrating the filling material being injected into the container.

FIG. 6 is a view schematically illustrating the container filled with the injected filling material being caused to indwell between adjacent spinous processes.

FIGS. 7A and 7B are views illustrating the operation of the filling material according to the embodiment. FIG. 7A is a view illustrating a state before the filling material is injected into the container, and FIG. 7B is a view illustrating a state where the filling material begins to be injected into the container.

FIGS. 8A and 8B are views illustrating the operation of the filling material according to the embodiment. FIG. 8A is a view illustrating a state where the filling material expands the container, and FIG. 8B is a view illustrating a state where the inside of the container is fully filled with the filling material.

FIGS. 9A and 9B are pictures of X-ray images. FIG. 9A is a captured X-ray image showing the first filling material and the second filling material, and FIG. 9B is a partially enlarged view of a portion on the captured X-ray image.

FIGS. 10A and 10B are views illustrating the posture of a subject when the medical device and filling material embodiment are used. FIG. 10A is a view illustrating the subject lying down, and FIG. 10B is a view illustrating the subject sitting down.

FIGS. 11A and 11B are view illustrating a modification of the puncture member included in the medical device. FIG. 11A is a side view of the modified puncture member, and FIG. 11B is a perspective view illustrating an enlarged needle tip of the modified puncture member.

FIGS. 12A, 12B and 12C are views illustrating a modification of the puncture member included in the medical device. FIG. 12A is a side view of the modified puncture member, FIG. 12B is a perspective view illustrating an enlarged portion 10B of FIG. 12A, and FIG. 12C is a view illustrating a state where the spring is compressed.

FIG. 13 is a graph illustrating a relationship between puncture resistance and puncture depth.

FIGS. 14A, 14B and 14C are views illustrating a modification example of the flexible container. FIG. 14A is a view illustrating a state before the filling material is injected into the container, FIG. 14B is a view illustrating a state where the filling material is injected into the container, and FIG. 14C is a view illustrating a state after the inside of the container is filled with the filling material.

FIG. 15 is a partial cross-sectional view briefly illustrating an overall configuration of a catheter system used for injecting the filling material into the container.

FIGS. 16A, 16B and 16C are views illustrating the operation of the catheter system. FIG. 16A is a view illustrating an injection position where the catheter system starts to inject the filling material into the container, FIG. 16B is a view illustrating a state where the injection position of the filling material is changed from the injection position illustrated in FIG. 16A, and FIG. 16C is a view illustrating a state where the injection position of the filling material is changed from the injection position illustrated in FIG. 16B.

FIG. 17 is a partial cross-sectional view briefly illustrating an overall configuration according to a modification example of a catheter system used for injecting the filling material into the container.

FIGS. 18A, 18B, and 18C are views illustrating an assisting device used for broadening a gap between adjacent spinous processes. FIG. 18A is a view illustrating an overall configuration of the assisting device, FIG. 18B is a view illustrating an enlarged distal portion of the assisting device, and FIG. 18C is a view illustrating the enlarged distal portion of the assisting device when an expansion portion included in the assisting device expands.

FIGS. 19A, 19B, and 19C are views illustrating a modification example of the assisting device used for broadening a gap between adjacent spinous processes. FIG. 19A is a view illustrating an overall configuration of the assisting device, FIG. 19B is a view illustrating an enlarged distal portion of the assisting device, and FIG. 19C is a view illustrating the enlarged distal portion of the assisting device when an expansion portion included in the assisting device expands.

FIG. 20 is a view illustrating an example of the assisting device illustrated in FIG. 19 being used.

### DETAILED DESCRIPTION

Set forth below is a detailed description of embodiments of a medical device for injecting filling material, a filling material and a method for injecting a filling material representing examples of the inventive medical device, filling material and method disclosed here. In some cases, dimensional proportions in the drawings may be exaggerated and different from actual proportions for convenience of description.

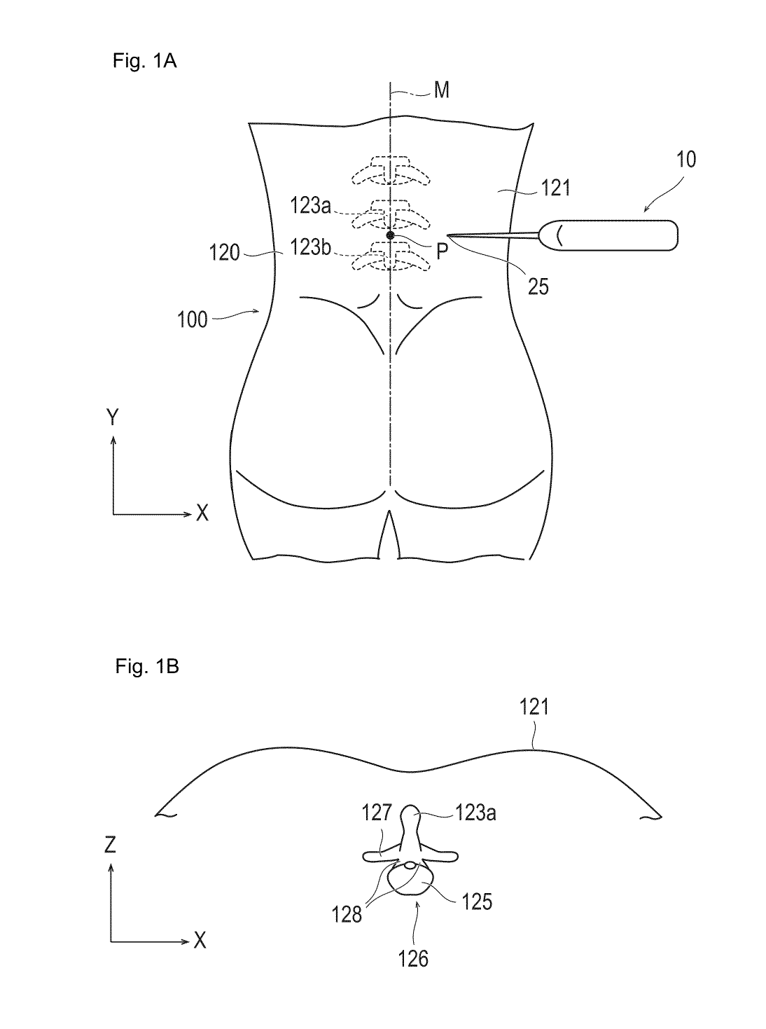

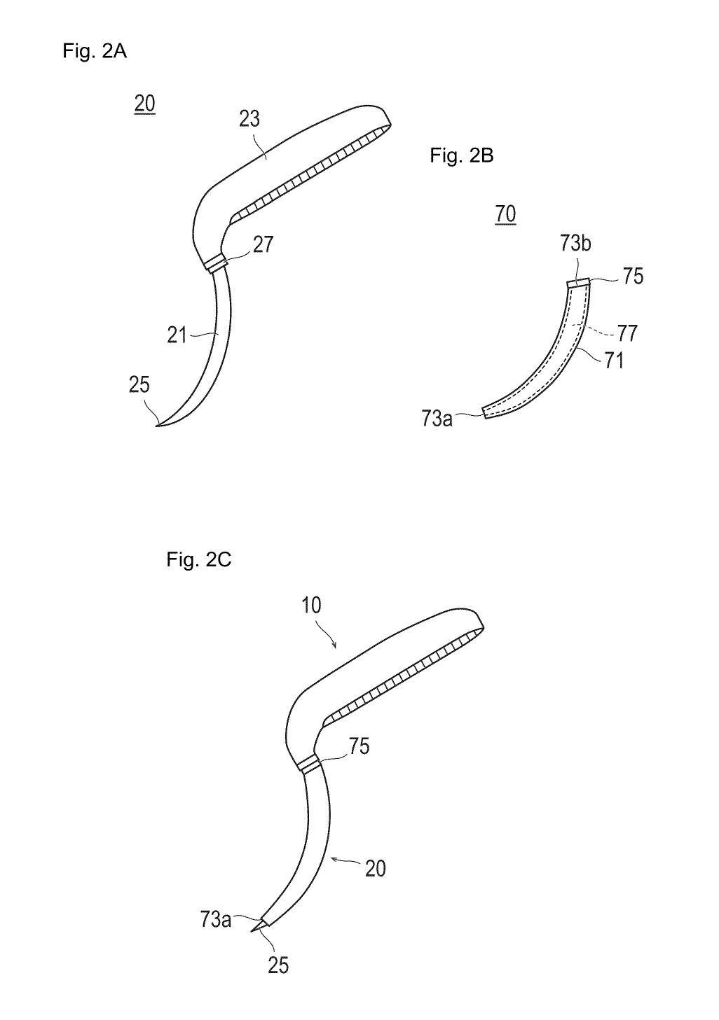

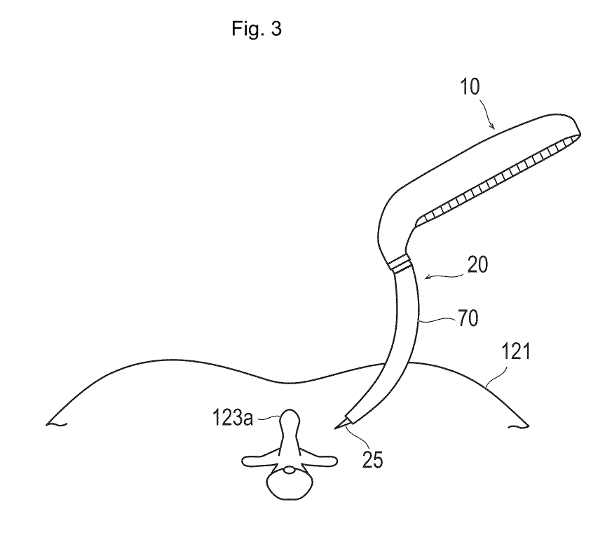

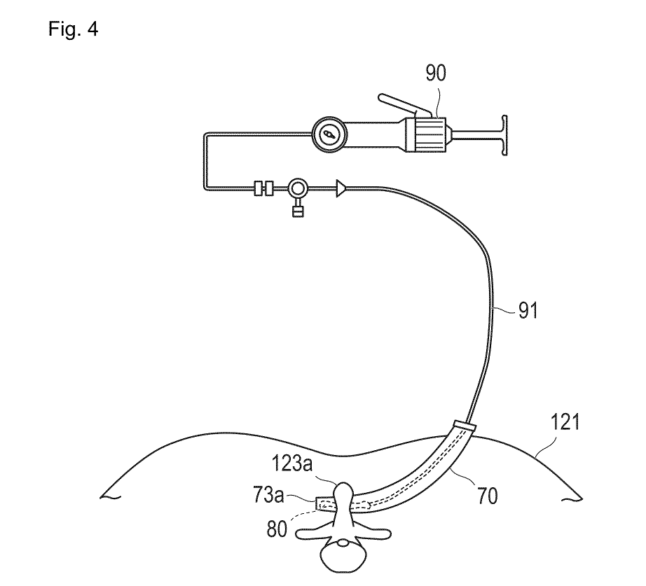

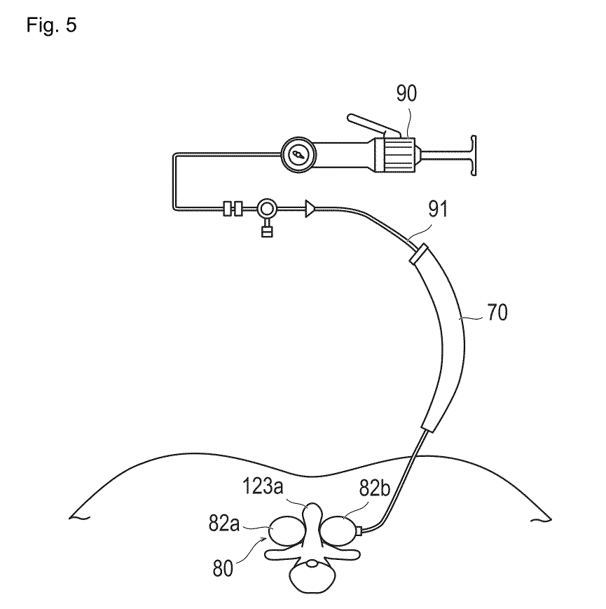

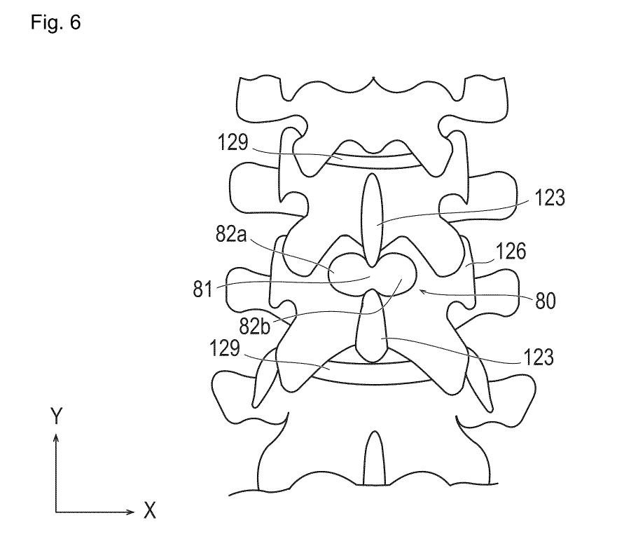

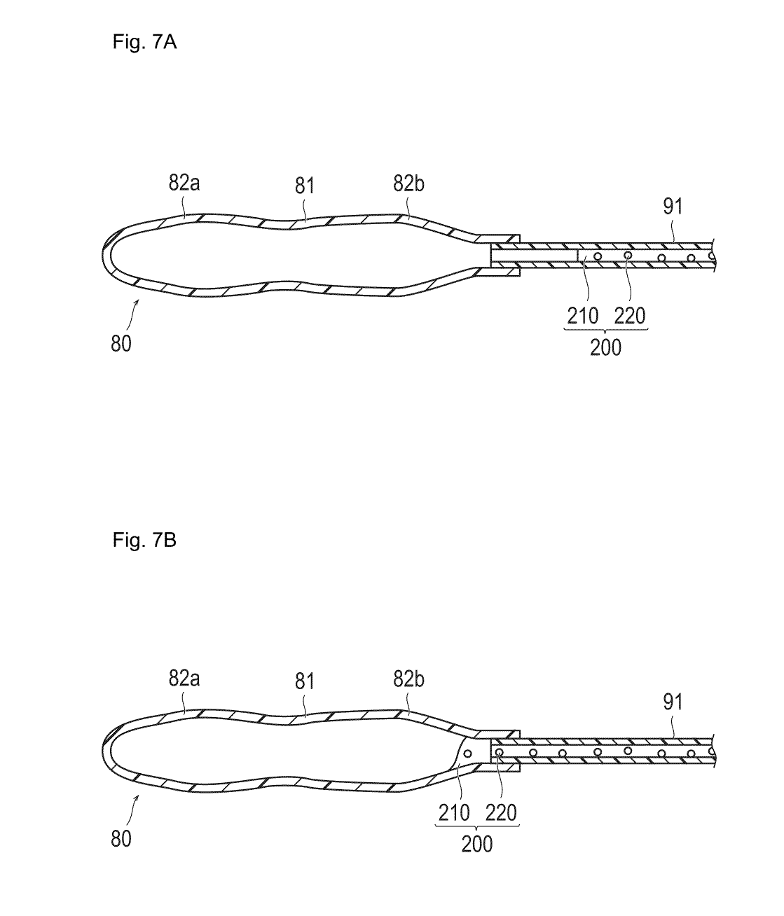

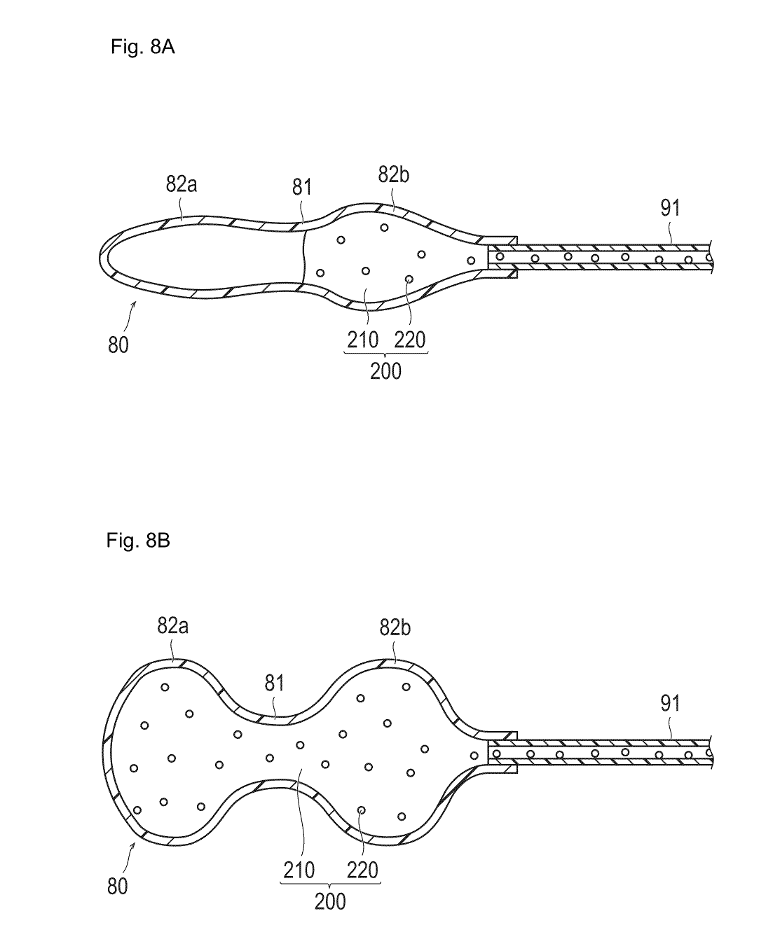

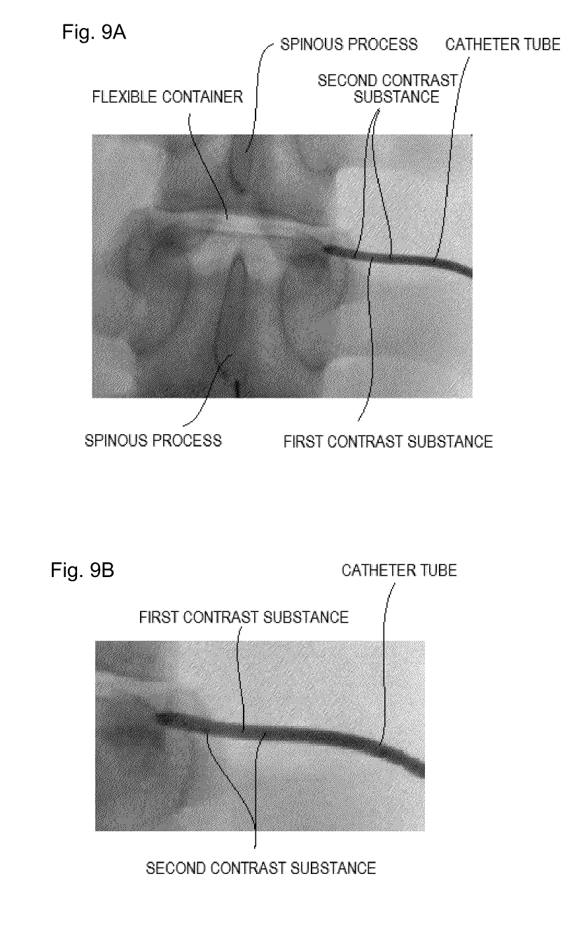

FIGS. 1A and 1B are views illustrating a living body for the manually operated device and filling material according to an embodiment of this application. FIGS. 2A, 2B and 2C are views illustrating a manually operated medical device that uses the filling material according to the embodiment. FIG. 3 is a view schematically illustrating the needle portion of the puncture member puncturing the living body. FIG. 4 is a view schematically illustrating a flexible container being introduced into the living body by using the guide member. FIG. 5 is a view schematically illustrating the filling material being injected into the container. FIG. 6 is a view schematically illustrating the container filled with the injected filling material being caused to indwell between adjacent spinous processes. FIGS. 7A and 7B are views illustrating the operation of the filling material according to the embodiment. FIGS. 8A and 8B are views illustrating the operation of the filling material according to the embodiment. FIGS. 9A and 9B are captured X-ray images.

In the present embodiment, the filling material will be described by using an example of maintaining a gap between adjacent spinous processes by injecting the filling material into a flexible container introduced between the adjacent spinous processes in a living body, and by causing the container to indwell between the spinous processes.

First, referring to FIGS. 1, 5, and 6, the spinous processes where the flexible container is caused to indwell, and a treatment target disease will be briefly described.

FIG. 1A is a view schematically illustrating a perspective view of a lumbar vertebra from a back side of the living body. FIGS. 1B and 5 are views schematically illustrating a cross section (horizontal cross section) of the living body in a direction orthogonal to an array direction of the spinous processes (i.e., the extending direction of the spine) which partially configure the lumbar vertebra. FIG. 6 is a view illustrating an enlarged site of the spinous processes illustrated in FIG. 1A. The X-axis in each of the drawings represents the direction orthogonal to the array direction of the spinous processes, the Y-axis represents the array direction of the spinous processes (i.e., the extending direction of the spine), and the Z-axis represents a thickness direction of the living body.

As illustrated in FIGS. 1A and 6, multiple lumbar vertebrae 126 are arranged along the extending direction of the spine in a back 121 of a living body 120 of a subject 100. As illustrated in FIG. 1B, the lumbar vertebrae 126 have a configuration in which a vertebral body 125 in the front half and a lamina of vertebral arch 127 in the rear half are interlocked with each other via a pedicle of vertebral arch 128. Various processes such as a spinous process 123, a costal (transverse) process, a superior articular process, an inferior articular process, and the like are formed in the lamina of vertebral arch 127. The lumbar vertebrae 126 normally have a slightly curved forward shape (i.e., towards the front of the living body 120). In addition, as illustrated in FIG. 6, the adjacent lumbar vertebrae 126 are interlocked with each other via an intervertebral disk (discus intervertebralis) 129. A certain lumbar vertebra and a lumbar vertebra adjacent to the lumbar vertebra do not deviate from each other because of an intervertebral joint or the like which is between the intervertebral disk 129, the superior articular process, and the inferior articular process.

For example, a stress fracture may arise when repeated loads are applied to the lumbar vertebrae 126 by sporting activities. The stress fracture sometimes results in a spondylolysis in which the vertebral body 125 and the lamina of vertebral arch 127 are separated from each other in an area of the pedicle of vertebral arch 128, or a spondylolisthesis in which the lumbar vertebrae 126 on the upper side deviate (i.e., displace upwardly) since the lumbar vertebrae 126 become less likely to be fixed due to the deformed intervertebral joint or the degenerated intervertebral disk 129. Due to this spondylolysis or spondylolisthesis, the ligament arranged around the lumbar vertebra degenerates (e.g., with old age), thereby causing spinal canal stenosis. Consequently, sometimes a symptom of lumbar spinal canal stenosis called intermittent claudication occurs. The spinal canal stenosis can be suppressed by indwelling an implant that functions as a spacer between two adjacent spinous processes 123a and 123b. For example, the implant can include a very rigid metal material. However, it is not preferable to use this rigid implant because the use of the implant is invasive to the living body. The present embodiment thus instead indwells a flexible container 80, having an injected filling material 200 (to be described later), between adjacent spinous processes 123a and 123b so that the container 80 functions as the implant. In this manner, the flexible container exhibits less invasive aspects for the living body compared to the metal-made implant in the related art.

Next, the flexible container will be described.

As illustrated in FIG. 4, the flexible container 80 (hereinafter, also referred to as a “container”) is contracted (i.e., not expanded or collapsed) in a state before the filling material 200 is injected. The container 80 is configured to expand when the filling material is injected and deform as illustrated in FIGS. 5 and 6.

The container 80 has a body portion 81 which is arranged between the adjacent spinous processes 123a and 123b, and two end portions 82a and 82b which are integrally disposed in the body portion 81 (i.e., the container 80 possesses a distal portion 82a, an intermediate portion 81, and a proximal portion 82b). The end portion located on the left side in FIG. 5 is referred to in this description as the distal side end portion 82a, and the end portion located on the right side of FIG. 5 is referred to as the proximal side end portion 82b.

As illustrated in FIG. 6, the flexible container 80 is shaped in advance so that both end portions 82a and 82b respectively form a convexly protruding shape in an expanded and deformed state. The body portion 81 is expanded and deformed while maintaining a substantially linear shape to support the adjacent spinous processes 123a and 123b so as to maintain a gap between the adjacent spinous processes 123a and 123b. An outer shape of the flexible container 80 before and after the container 80 is expanded and deformed is not limited to the shape illustrated in FIG. 6. The container 80 just has to be able to be arranged between the adjacent spinous processes 123a and 123b to maintain the gap between the adjacent spinous processes 123a and 123b at a predetermined distance. However, if the container 80 is configured so that both end portions 82a and 82b protrude in an array direction of the spinous processes (i.e., the Z-axis direction in FIG. 6) after the container 80 is expanded and deformed as illustrated, the spinous process becomes interposed between both end portions 82a and 82b. Therefore, it is possible to efficiently prevent the container 80 from being misaligned.

The material of the flexible container 80 is not particularly limited as long as the material can withstand external pressure applied by the spinous process or external pressure resulting from the movement of vertebral body, and as long as the container 80 can be expanded and deformed by injecting the filling material 200 (i.e., the flexible container 80 is expandable). Examples of preferred materials to use for the flexible container 80 include thermoplastic elastomers such as vinyl chloride, polyurethane elastomers, styrene-ethylene-butylene-styrene copolymer (SEBS), styrene-ethylene-propylene-styrene copolymer (SEPS), and the like, thermoplastic resins such as nylon, PET, and the like, or thermosetting resins such as rubber, silicone elastomers, and the like. It is particularly preferable to use porous materials such as nonwoven fabric, woven fabric, knitted fabric, ePTFE, and the like. In addition, it is also possible to use a proper combination of these materials.

Next, the filling material 200 will be described.

As described above, the filling material 200 according to the present embodiment is injected into the expandable and flexible container 80 while the flexible container 80 is within the living body 120 of the subject 100. As illustrated in FIGS. 7A and 7B, the filling material 200 is a mixture of a first contrast substance 210 and a second contrast substance 220.

The first contrast substance 210 includes X-ray contrast properties and fluidity (i.e., the first contrast substance 210 is flowable, i.e., is able to flow). The second contrast substance 220 is insoluble in the first contrast substance 210 and possesses X-ray contrast properties which are higher than those of the first contrast substance 210 (i.e., the second contrast substance 220 possesses higher contrast X-ray contrast properties than the first contrast substance 210). The second contrast substance 220 is contained in the filling material 200 in a state where the second contrast substance 220 is mixed in the first contrast substance 210. In the present embodiment, the description that the second contrast substance 220 is mixed in the first contrast substance 210 means that the second contrast substance 220 is in an insoluble state in the first contrast substance 210 and is present in a state of maintaining a predetermined shape.

First, the first contrast substance will be described.

The first contrast substance 210 can be configured to have a material including fluidity, such as liquid, gel, and the like.

In the filling material 200 according to the present embodiment, the first contrast substance 210 is configured to be cured with the lapse of time (i.e., the first contrast substance 210 cures after a predetermined period of time). A curable first contrast substance 210 provides the following advantages. As described above, the flexible container 80 functions as the implant for maintaining a gap between the adjacent spinous processes 123a and 123b when the inside of the container 80 is filled with the filling material 200. Therefore, when the filling material 200 cures within the indwelled container 80, misalignment of the container 80 is further prevented and the container 80 can maintain the gap between the spinous processes stably over a long period of time.

In order to prevent the spinous process or the living body organ around the spinous process from being damaged by the cured first contrast substance 210, it is preferable to set the hardness or the Young's modulus of the cured first contrast substance 210 to have a predetermined magnitude. For example, it is preferable to set the hardness of the cured first contrast substance 210 so that Shore A hardness is 25 to 100 and Shore D hardness is 30 to 90. More preferably the Shore A hardness may be between 25 and 95, and the Shore D hardness may be between 40 and 60. In addition, it is preferable to set the Young's modulus of the cured first contrast substance 210 to be 0.005 MPa to 3600 MPa, and more preferably 0.5 MPa to 100 MPa.

Preferably, the first contrast substance 210 according to the present embodiment has at least one of the following characteristics: (a) safe for patients; (b) no or little damage to biological tissues; (c) curable at a temperature close to a patient's body temperature (approximately 35° C. to 42° C. or 95° F. to 107° F.); (d) a cured shape can be maintained without any contraction or expansion; (e) a cure time of under one minute to 60 minutes (i.e., one hour) after injection, preferably within five minutes to 30 minutes, and more preferably within 10 minutes; or (f) water, a buffer solution, a physiological saline solution, a contrast agent, or oils and fats such as olive oil, castor oil, and the like are used as a solvent in the first contrast substance 210.

Specific examples of a curing agent include (a) two-liquid mixture cross-linked polymer; (b) hot melt adhesive; (c) urethane elastomer; (d) a photo-curable resin; (e) an acrylic resin; (f) bone cement; or (g) a solution which is crystallized by external stimulus.

When the curing agent is a two-liquid mixture cross-linked polymer, it is preferable to use a combination of aromatic diepoxide resin or aliphatic diepoxide resin and amine compound, or alternatively a combination of liquefied polyorganosiloxane having a reactive group, a crosslinking agent, and a curing catalyst. The polyorganosiloxane includes materials in which a functional group of main chain siloxane has a methyl group, a vinyl group, a hydroxyl group, a ketone group, and the like. However, it is preferable to use a material having the vinyl group as an additional type. In addition, as the crosslinking agent, polysiloxane having a hydrogen group or the like is used. As the curing catalyst, platinum compound, organometallic compound, or the like is used. Furthermore, for example, a reaction control agent, reinforcing silica, other filling materials and/or additives may be contained.

Examples of the hot melt adhesive curing agent include a combination of a material curable by reaction with water and water, or an ethylene-vinyl acetate copolymer (EVA) system, a polyolefin (PO) system, a polyamide (PA) system, synthetic rubber (SR) system, acrylic (ACR) system, a polyurethane (PUR, moisture-curing type) system, and the like.

When the curing agent is the urethane elastomer, it is preferable to use polymer derived from polyol and aromatic polyisocyanate.

When the curing agent is the photo-curable resin, photo-polymerizable monomer includes acrylic ester, methacrylic acid ester, ethylenically unsaturated carboxylic acid, or the like. If necessary, it is possible to use a polymerization accelerator, a crosslinking agent, a photoinitiator, or the like.

Examples of the acrylic resin curing agent include those which are obtained by using a known method to polymerize monomers such as methyl(meth)acrylate, ethyl(meth)acrylate, butyl(meth)acrylate, n-hexyl(meth)acrylate, cyclohexyl(meth)acrylate, ethylhexyl(meth)2-acrylate, n-octyl(meth)acrylate, nonyl(meth)acrylate, decyl(meth)acrylate, (meth)acrylate, glycidyl(meth)acrylate, vinyl acetate, styrene, α-methyl styrene, (meth)acrylamide, (meth)acrylonitrile, or the like.

Examples of the bone cement curing agent include bone cement produced by mixing powder such as polymethyl methacrylate, methyl methacrylate-styrene copolymer, benzoyl peroxide, barium sulfate, or the like with a solvent containing methyl methacrylate, N,N-dimethyl-p-toluidine, hydroquinone, or the like. Alternatively, a solvent may be mixed with dental cement cured by an acid-base reaction between zinc oxide and phosphoric acid, sodium alginate, sodium phosphate, calcium chloride, or the like. In this manner, it is also possible to use an organic-inorganic composite material or the like in which organic sodium alginate and inorganic calcium phosphate are produced.

Examples of the solution crystallized by external stimulus include an aqueous solution obtained by dissolving sodium acetate, sodium chloride, or the like. The external stimulus includes physical shock, heat, light, electricity, ultrasound, or the like.

Next, the second contrast substance will be described.

The second contrast substance 220 can contain a solid contrast agent (e.g., the second contrast substance 220 can be a solid). For example, the solid contrast agent can be barium sulfate, calcium tungstate, strontium oxide, zirconium oxide, bismuth oxide, lanthanum oxide, zinc oxide, zirconium phosphate, basic bismuth carbonate, bismuth sulfate, zirconium silicate, ytterbium fluoride, strontium glass, and barium glass. The second contrast substance 220 concentration in the first contract substance 210 is preferably between 1% and 20%. The solid contrast agent contained in the second contrast substance 220 is preferably a metal material which is relatively highly insoluble in the first contrast substance 210 among the above-described contrast agents.

In the present embodiment, the second contrast substance 220 is configured to be scattered in a particle shape (i.e., solid particles) in the first contrast substance 210 (e.g., see FIGS. 7A to 8B). The second contrast substance 220 being scattered in the first contrast substance 210 means that the respective second contrast substances 220 mixed in the first contrast substances 210 are separated from each other by a predetermined distance without being in a state where the respective contrast substances are close and connected to each other.

The particle size of the second contrast substance 220 is not particularly limited as long as the second contrast substance 220 is visible on a captured X-ray image. However, it is preferable to use particle sizes of 0.007 mm or larger and smaller than 2.0 mm. The reason is as follows.

As described in the present embodiment, when an operator uses his or her manual skills for maintain the gap between the adjacent spinous processes, he or she generally carries out the work while confirming an X-ray image projected on an image display monitor. The first contrast substance 210 and the second contrast substance 220 are projected on the image display monitor. However, if the particle size of the second contrast substance 220 is too small, it is not possible to visually recognize the second contrast substance 220 when observing the X-ray image. Therefore, it becomes necessary to form the particle size of the second contrast substance 220 so as to be large to some extent (i.e., large enough to be visible on the X-ray image). However, if the particle size is excessively large, there is a possibility that the filling material 200 may be inhibited from flowing due to a relationship with an inner diameter of a catheter tube 91 (refer to FIG. 7) used in injecting the filling material 200. The inner diameter of the catheter tube 91 is not particularly limited, but according to the present embodiment, the inner diameter is designed to be 2 mm in view of the relationship between the fluidity of the filling material 200 and the wall face friction resistance of the catheter tube 91. Therefore, it is preferable to use a second contrast substance 220 with a particle size smaller than 2 mm. On the other hand, when the filling material 200 is observed on a general-purpose image display monitor (50 inches, aspect ratio of 16:9), if the particle size is 0.007 mm, the particle size can be enlarged up to approximately 0.2 mm for display. Although it depends on a distance between an observer and the image display monitor, if the second contrast substance 220 is displayed with a size of 0.2 mm, the observer can identify the second contrast substance 220 with the naked eye and confirm the state of the filling material 200 (to be further described below). Therefore, when the second contrast substance 220 is in a particle shape, it is preferable to set the particle size to 0.007 mm or larger and smaller than 2.0 mm. In order to enable the observer to reliably recognize the second contrast substance 220 with the naked eye, for example, it is preferable to set the particle size of the second contrast substance 220 to 0.2 mm or larger and smaller than 2.0 mm, and more preferably 0.5 mm or larger and smaller than 2.0 mm.

Next, a brightness difference between the first contrast substance 210 and the second contrast substance 220 on an X-ray image will be described.

In order to visually recognize the second contrast substance 220 mixed in the first contrast substance 210 on the X-ray image, a predetermined brightness difference is required between the first contrast substance 210 and the second contrast substance 220. The brightness difference represents a difference of brightness between the first contrast substance 210 and the second contrast substance 220 on the X-ray image. As the brightness difference increases, the second contrast substance 220 becomes darker than the color of the first contrast substance 210 (e.g., which is a background color). When the brightness difference between the first contrast substance 210 and the second contrast substance 220 is relatively small, the respective contrast substances 210 and 220 are projected on the X-ray image and have approximately the same color depth. Consequently, it becomes difficult to identify the respective contrast substances 210 and 220 with the naked eye.

Visibility of the respective contrast substances 210 and 220 based on the brightness difference is affected by an observer's eyesight, brightness of the surrounding environment during observation, and the like. However, if the brightness difference between the first contrast substance 210 and the second contrast substance 220 is five or more as can be confirmed from the result of the application example (to be described later), it is possible to clearly identify the second contrast substance 220 with the naked eye. Therefore, it is preferable to adjust the brightness difference so as to be at least five on the below described brightness scale. As will be described later, in order to enable an observer to more reliably identify the second contrast substance 220 on the X-ray image with the naked eye, it is preferable to set the brightness difference to 20 or more on the below described brightness scale.

As a method of manufacturing the filling material 200 mixture of the first contrast substance 210 and the second contrast substance 220, for example, it is possible to employ a method in which the second contrast substance 220 whose particle size is previously adjusted is added to the first contrast substance 210 which is adjusted into a flowable state. It is also possible to use a method in which the second contrast substance 220 adjusted into a powder shape is sieved into the first contrast substance, or a method in which the second contrast substance 220 having a predetermined particle size is selected and added to the first contrast substance 210.

The brightness of the respective contrast substances 210 and 220 can be adjusted by adjusting the amount of contrast components contained in the contrast substance, or by adjusting the mixing ratio of the contrast components for each contrast substance. The X-ray contrast properties of the flexible container 80 is set to be lower than the X-ray contrast properties of each of the first contrast substance 210 and the second contrast substance 220 which configure the filling material 200 (i.e., the flexible container 80 has lower contrast X-ray contrast properties than the filling material 200). The setting is made in order to project the filling material 200 injected into the container 80 after transmitting X-rays through the container 80 during X-ray photography while work for injecting the filling material 200 is carried out.

Next, a medical device used for introducing the flexible container into the adjacent spinous processes will be described.

As illustrated in FIG. 2A to 2C, a medical device 10 has a puncture member 20 and a guide member 70 used while being integrally assembled to the puncture member 20.

As illustrated in FIG. 2A, the puncture member 20 has a needle portion 21 which is curved at a predetermined curvature, and a main body portion 23 which is disposed on the proximal side of the needle portion 21.

The main body portion 23 is configured so that a user can grip the main body portion 23 when the user uses the medical device 10. The needle portion 21 is configured so that the outer diameter gradually decreases from the proximal side toward the distal side (i.e., the needle portion 21 tapers towards the needle tip 25 at the distal-most end).

A needle tip 25 located at the distal end of the needle portion 21 has a thinly and sharply pointed shape. The needle portion 21 can be configured to be connectable to and detachable from the main body portion 23. To be connectable and detachable from the main body portion 23, the proximal end of the needle portion 21 may be mechanically connected to the main body portion 23 by means of screwing, fitting, or the like.

A material for configuring the needle portion 21 is not particularly limited as long as the material can puncture a living body. For example, the needle portion 21 material can include metal materials such as SUS, titanium, magnesium, chromium, cobalt, nickel, aluminum, gold, silver, copper, iron, and the like, or resin materials such as polyether ether ketone (PEEK), polycarbonate (PC), polycarbonate urethane (PCU), reinforced polyphenylene (SRP), carbon or glass fiber reinforced polymer, and the like.

Although particularly not limited, the main body portion 23 material may be a metal material, a rigid plastic material, or the like.

As illustrated in FIG. 2B, the guide member 70 can be configured to have an insertion portion 71 which is curved at substantially the same curvature as that of the needle portion 21 of the puncture member 20, an opening portion 73a on the distal side of the insertion portion 71, an opening portion 73b on the proximal side of the insertion portion 71, a connection portion 75 which is disposed on the proximal side of the insertion portion 71, and which can be connected to and detached from the main body portion 23 of the puncture member 10, and a lumen 77 which is formed inside the insertion portion 71.

As illustrated in FIG. 2C, the needle portion 21 of the puncture member 20 can be inserted into the lumen 77 of the guide member 70. The length of the insertion portion 71 of the guide member 70 is shorter than the length of the needle portion 21. Therefore, when the needle portion 21 is inserted into the lumen 77, the needle tip 25 of the needle portion 21 is exposed from the opening portion 73a on the distal side of the guide member 70 by an amount of a predetermined length.

In addition, the needle portion 21 is inserted into the lumen 77. In this manner, the puncture member 20 and the guide member 70 can be assembled together via an interlock portion 27 disposed in the puncture member 20 and the connection portion 75 disposed in the guide member 70. For example, the connection portion 75 of the guide member 70 can be mechanically connected to the interlock portion 27 of the puncture member 20 by means of fitting. However, the configuration is not particularly limited as long as the guide member 70 can be connected to and detached from the puncture member 20.

For example, as a material for configuring the insertion portion 71 of the guide member 70, it is possible to use the same material as that of the needle portion 21 of the previously described puncture member 20.

Next, the process flow of manually operating the medical device and the flexible container will be described.

First, as illustrated in FIG. 2C, the puncture member 20 and the guide member 70 are assembled together to form the medical device 10.

Next, as illustrated in FIG. 3, the needle tip 25 of the needle portion 21 of the puncture member 20 punctures the back 121 of the living body 120 at a predetermined position. FIG. 1A illustrates an orientation of the needle tip 25 of the needle portion 21 of the puncture member 20 before the puncture is performed. The needle tip 25 is moved close to the back 121 of the subject 100 in the state illustrated in FIG. 1A, thereby causing the needle tip 25 to pierce the back 121 at the predetermined position.

After the needle tip 25 pierces the back 121, the operator moves the needle tip 25 toward the spinous process 123a. FIG. 1A illustrates an example indwelling target position P where the flexible container 80 is caused to indwell. A piercing position or a puncture route is not particularly limited, and can be changed as long as the needle tip 25 can reach at least the vicinity of the indwelling target position P. As illustrated in FIG. 1A, according to the present embodiment, the indwelling target position P located on a median line M of the living body (i.e., the median of the spine). However, the indwelling target position P can be changed depending on diseases or disease sites of the subject 100, and is not limited to the illustrated position.

The puncture member 20 is moved until the needle tip 25 of the puncture member 20 and the distal opening portion 73a of the guide member 70 reach the vicinity of the spinous process 123a. According to the present embodiment, the flexible container 80 is arranged in the vicinity of the central position in the lateral direction of the spinous process 123a. Accordingly, the puncture member 20 is moved to a position where the needle tip 25 and the distal opening portion 73a of the guide member 70 reach the proximal side (left side of FIG. 4) in the puncture direction after crossing the spinous process 123a. Next, the puncture member 20 is separated from the guide member 70. Then, the puncture member 20 is removed from the lumen 77 of the guide member 70. Thereafter, as illustrated in FIG. 4, the flexible container 80 is guided to the vicinity of the spinous process 123a via the lumen 77 of the guide member 70.

Next, the guide member 70 is pulled back in the proximal end direction, and the flexible container 80 is exposed from the guide member 70. In this case, the distal end of the guide member 70 may be brought into a puncture state inside the living body 120, or may be brought into a state of being completely removed out from the living body 120 as illustrated in FIG. 5.

Next, the filling material 200 is injected into the flexible container 80.

When the filling material 200 is injected into the flexible container 80, it is possible to use a known fluid supply device 90 such as a syringe pump which can pump a fluid. The flexible container 80 and the fluid supply device 90 can be interlocked with each other in advance by using a catheter tube 91 or a similar known tube through which the fluid can flow. The flexible container 80 and the catheter tube 91 can be connectable and separable from each other, for example, by a method such as fitting, screwing, cutting, and the like.

The X-ray contrast properties of the above-described catheter tube 91 are set to be lower than those of each of the first contrast substance 210 and the second contrast substance 220 which constitute the filling material 200. The lower contrast is used in order to project the filling material 200 present inside the catheter tube 91 after transmitting X-rays through the catheter tube 91 during X-ray photography while work for injecting the filling material 200 is carried out (i.e., so that the filling material 200 can be viewed on the X-ray images).

If the distal end of the guide member 70 is arranged on the further proximal side (right side in the drawing) from the spinous process 123a instead of being positioned on the further distal side from the spinous process as illustrated in FIG. 4, a mandrel is inserted into the flexible container 80, and the catheter tube 91 is pushed forward in the distal end direction. In this manner, the flexible container 80 is also enabled to reach the vicinity of the spinous process 123a without operating the guide member 70 so as to be pulled back in the proximal end direction.

The filling material 200 is injected into the flexible container 80, thereby expanding the flexible container 80. Thereafter, the catheter tube 91 is separated from the container 80, and the catheter tube 91 is removed out from the living body 120 of the subject 100. A sealing member, a valve, or the like which prevents the filling material 200 from leaking out when the catheter tube 91 is separated can be disposed in an interlock portion between the flexible container 80 and the catheter tube 91.

As illustrated in FIG. 6, the flexible container 80 is caused to indwell when filled with the filling material 200. A gap between the adjacent spinous processes 123a and 123b is maintained by the flexible container 80 indwelled in this manner. It is thus possible to obtain an effective treatment effect for a symptom of the lumbar spinal canal stenosis.

Next, the operation of the filling material according to the present embodiment will be described with reference to FIGS. 7A to 8B.

FIGS. 7A to 8B schematically illustrate an X-ray image obtained by imaging the flexible container 80 before, during, and after the filling material 200 is injected into the flexible container 80 within the living body 120. A known X-ray imaging apparatus can be used for the medical practices discussed below.

FIG. 7A illustrates an initial stage of the injection work. It is possible to confirm that the filling material 200 moves inside the catheter tube 91 in a state where the second contrast substance 220 is mixed in the flowable first contrast substance 210. In order to facilitate understanding, the flexible container 80 and the catheter tube 91 are illustrated by using a cross section view in FIGS. 7A to 8B. In addition, the first contrast substance 210 is not colored, and the second contrast substance 220 is illustrated by an open circle. However, the respective contrast substances are projected onto an X-ray image as a grayscale image whose basic tone is a black color.

FIG. 7B illustrates a state where the work for injecting the filling material 200 is continuously carried out (i.e., the filling material 200 is continuously being introduced into the flexible container 80 and FIG. 7B illustrates the flexible container 80 beginning to be filled). By referring to the X-ray image as illustrated in FIG. 7B, it is possible to confirm a state where the filling material 200 containing the first contrast substance 210 and the second contrast substance 220 is injected into the flexible container 80. In the filling material 200 according to the present embodiment, the second contrast substance 220 is scattered in a particle shape in the first contrast substance 210. Therefore, it is possible to confirm a flowing state of the filling material 200 by observing each movement of the second contrast substance 220, and it is possible to confirm the flowing state of the filling material 200 by perspectively viewing the whole X-ray image (i.e., the flow rate of the second contrast substance 220 is easier to detect and so the flowing state of the filling material 200 can be observed).

As illustrated in FIG. 8A, the container 80 is progressively expanded and deformed (e.g., the container 80 bulges outwardly at the end portions 82a, 82b) as the amount of the filling material 200 injected into the flexible container 80 increases.

After the inside of the flexible container 80 is full of the filling material 200, the work for injecting the filling material 200 is completed. The completion of the injection work can be determined based on the captured X-ray image.

Here, when a filling material in the related art which does not contain the second contrast substance 220 is used, the second contrast substance 220 cannot be projected on to the X-ray image. In addition, the contrast properties of the filling material used in the related art are entirely uniformly adjusted so that uneven color depth (i.e., a brightness difference) does not occur on the X-ray image. Therefore, when the filling material in the related art is used, regardless of a change in the amount of the filling material injected into the flexible container, the filling material is projected as a grayscale image with entirely uniform brightness. Here, as a method of confirming a change in an injection state of the filling material, for example, it is conceivable to employ a determination method based on whether or not the filling material flows inside the catheter tube, or a determination method based on the amount of the filling material injected into the container, that is, an expanded degree of the container.

In the above-described methods, according to the determination method after confirming the flow of the filling material inside the catheter tube, the filling material is projected with uniform brightness. Consequently, it is not possible to clearly determine whether or not the filling material flows (i.e., the flow rate of the filling material cannot be determined). It is thus not possible to recognize a change in the injection state of the filling material. On the other hand, according to the determination method after confirming the expanded degree of the container, it is possible to confirm a change in an injection state based on a change in the outer shape of the container or the amount of the filling material injected into the container from the initial stage when the filling material starts to be injected until the container expands to some degree. However, if the container expands to some degree and a deformation amount of the outer shape of the container or the amount of the filling material injected into the container decreases per unit time, it is not easy to recognize the change only by observing the X-ray image (i.e., the rate of change of the container volume cannot be determined). In particular, when filling material having relatively higher viscosity is cured with the lapse of time or a smaller diameter catheter tube is used, the amount of the filling material injected into the container extremely decreases per unit time. Consequently, it becomes more difficult to recognize the above-described change only by observing the X-ray image. Therefore, when the container is expanded and deformed so as to have a predetermined outer shape, the work for injecting the filling material cannot be completed at the optimum timing for stopping the work. The injection work is thus inevitably completed in a state where the container is not sufficiently expanded, or the injection work is inevitably completed in a state where the filling material is excessively injected (i.e., the filling material 200 is over injected or under injected into the flexible container 80).

Compared to the method of using the filling material in the related art as described above, in the method of using the filling material 200 according to the present embodiment, the injection work of the filling material 200 can be completed at the preferable timing (i.e., avoiding over-injecting or under-injecting the filling material 200 into the flexible container 80) by observing the second contrast substance 220 contained in the filling material 200.

For example, referring to the X-ray image illustrated in FIG. 8B, when the second contrast substance 220 does not move inside the catheter tube 91, the filling material 200 does not flow. Accordingly, it is possible to confirm that the inside of the container 80 is filled with (full of) the filling material 200 (i.e., the flexible container 80 is fully filled with the filling material 200, but is not overly filled with the filling material 200). Confirming that the inside of the flexible container 200 is filled allows for an accurate determination of when to stop injecting the filling material 200. On the other hand, when the second contrast substance 220 moves inside the catheter tube 91, the filling material 200 flows (i.e., observing the second contrast substance 220 continuing to move on the X-ray image confirms that the container 80 is not yet fully filled). Accordingly, it is possible to confirm that the inside of the container 80 is not yet filled with the filling material 200. Therefore, it is possible to determine whether to continuously carry out the injection work until a state where the filling material 200 does not flow.

As described above, according to the filling material 200 in the present embodiment, the X-ray contrast properties of the second contrast substance 220 mixed in the flowable first contrast substance 210 are higher than the X-ray contrast properties of the first contrast substance 210 (i.e., the second contrast substance 220 is higher contrast than the first contrast substance 220). Accordingly, it is possible to easily visually identify the first contrast substance 210 and the second contrast substance 220 on the X-ray image making it possible to recognize a flowing state of the whole filling material 200 by observing the movement of the second contrast substance 220. Accordingly, based on the observation result, it is possible to easily confirm the state when the filling material 200 has been fully injected into the container 80.

In addition, the brightness difference is set to five or more between the first contrast substance 210 and the second contrast substance 220. This allows the observer to more reliably identify the first contrast substance 210 and the second contrast substance 220, and to smoothly carry out the work for confirming an injection state of the filling material 200.

In addition, the second contrast substance 220 is scattered in a particle shape in the first contrast substance 210. Accordingly, it is possible to more easily observe each movement of the second contrast substance 220.

In addition, the particle size of the second contrast substance 220 is formed to be 0.007 mm or larger and smaller than 2.0 mm. Accordingly, it is possible to display the second contrast substance 220 using a visible size when the captured X-ray image is referred to, and it is possible to more conveniently and quickly carry out the work for confirming an injection state of the filling material 200.

In addition, the first contrast substance 210 is configured to be cured with the lapse of time (i.e., the first contrast substance 210 cures over time). Accordingly, it is possible to preferably apply the filling material 200 into the flexible container 80 to indwell the flexible container 80 in the living body to maintain and expand a gap between living body organs.

In addition, the second contrast substance 220 contains a metal material. Accordingly, it is possible to simply adjust the X-ray contrast properties so that the X-ray contrast properties of the second contrast substance 220 are higher than the X-ray contrast properties of the first contrast substance 210. In addition to this configuration, it is possible to adjust the X-ray contrast properties by adding an existing metal material having the X-ray contrast properties to the second contrast substance 220. Accordingly, it is possible to suppress an increase in manufacturing cost of the filling material 200.

The container 80 can be manually indwelled between the adjacent spinous processes 123a and 123b inside the living body 120 by injecting the filling material 200 into the flexible container 80. In this manner, it is possible to maintain a gap between the adjacent spinous processes 123a and 123b by using the container 80 having the injected filling material 200 (i.e., the container 80 is fully filled with the injected filling material 200). In addition, when the work is carried out in order to inject the filling material 200 into the flexible container 80, it is possible to confirm an expanded state of the container 80 by observing the movement of the second contrast substance 220. Accordingly, it is possible to obtain a guideline for recognizing a state of progress in the injection work, and it is possible to more conveniently and quickly use manual skills (i.e., manual operation) in order to maintain a gap between the spinous processes.

When the filling material 200 is injected into the flexible container 80 within the living body 120, it is possible to recognize a flowing state of the whole filling material 200 by observing the movement of the second contrast substance 220 mixed in the first contrast substance 210 (i.e., by observing the flow rate of the second contrast substance 220). Accordingly, it is possible to easily confirm a state where the filling material 200 is injected into the container 80.

Next, a result of an application example using the filling material according to the present invention will be described. Respective physical properties relating to the filling material employed in this application example are simply for illustrative purposes, and the filling material disclosed here is not limited to the illustrated configuration.

FIG. 9A illustrates an X-ray image (X-ray photograph) captured by an X-ray imaging apparatus during injection work, and FIG. 9B is a photograph illustrating a partially enlarged portion of a projected catheter tube in FIG. 9A.

According to the present application example, the filling material is injected into the flexible container arranged between the spinous processes. The present application example uses zirconium oxide prepared in a particle shape as the second contrast substance, and zirconium oxide scattered in vinyl polysiloxane as the first contrast substance. The first contrast substance has fluidity (i.e., flows). The second contrast substance is prepared so that each particle is between 0.007 mm and 2.0 mm, with a weight ratio of 10%. The second contrast substance is prepared so that a brightness difference between the first contrast substance and the second contrast substance is five or more on the brightness scale explained below.

Table 1 shows results of a visual inspection in which the second contrast substance is visually recognized based on the X-ray image obtained through the application example.

TABLE 1

| Particle Size A | Identification | Confirmation of |
|---|---|---|
| [mm] | Determination | Flowing State |
| 0.1 | 1 | ◯ |
| 0.2 | 2 | ◯ |
| 0.3 | 2 | ◯ |
| 0.4 | 2 | ◯ |
| 0.5 | 3 | ◯ |
| 0.6 | 3 | ◯ |
| 0.7 | 3 | ◯ |
| 0.8 | 3 | ◯ |
| 0.9 | 3 | ◯ |
| 1.0 | 3 | ◯ |
| 1.1 | 3 | ◯ |
| 1.2 | 3 | ◯ |
| 1.3 | 3 | ◯ |
| 1.4 | 3 | ◯ |
| 1.5 | 3 | ◯ |
| 1.6 | 3 | ◯ |
| 1.7 | 3 | ◯ |
| 1.8 | 3 | ◯ |
| 1.9 | 3 | ◯ |
| 2.0 | 3 | — |

A particle size A in Table 1 represents a particle size of the second contrast substance used in the application example. The “identification determination” shows results in which visibility of the second contrast substance is confirmed by observing the X-ray image illustrated in FIG. 9A. The results of the identification determination are as follows (i.e., the identification determination column values 1, 2, and 3 mean the following).

3: The second contrast substance can be clearly identified at a position of 0.7 m away from the X-ray image (normal use).

2: The second contrast substance can be identified at the position of 0.7 m away from the X-ray image (normal use).

1: The second contrast substance can be identified at a position of 0.3 m away from the X-ray image (i.e., the second contrast substance cannot be readily identified at a position of 0.7 m away from the X-ray image).

The “confirmation of the flowing state” in Table 1 represents results of visually confirming a state where the second contrast substance flows inside the catheter tube by observing the X-ray image. Those which can be confirmed are marked with “◯”. With regard to the second contrast substance having the respective particle sizes whose identification determination shows 1 to 3, the flowing state inside the catheter tube can be confirmed. Since the catheter tube in this example has an inner diameter is 2.0 mm, the flowing state inside the catheter tube cannot be confirmed when the second contrast substance has a particle size of 2.0 mm.

Table 2 shows a range of the particle sizes of the second contrast substance which can be identified when a general-purpose image display monitor (50 inches, aspect ratio of 16:9) is used.

TABLE 2

| Particle Size A [mm] | Particle Size B [mm] |
|---|---|
| 0.1 | 0.004 |
| 0.2 | 0.007 |
| 0.3 | 0.011 |
| 0.4 | 0.015 |
| 0.5 | 0.018 |
| 0.6 | 0.022 |
| 0.7 | 0.025 |
| 0.8 | 0.029 |
| 0.9 | 0.033 |
| 1.0 | 0.036 |
| 1.1 | 0.040 |
| 1.2 | 0.044 |
| 1.3 | 0.047 |
| 1.4 | 0.051 |
| 1.5 | 0.055 |
| 1.6 | 0.058 |
| 1.7 | 0.062 |
| 1.8 | 0.065 |
| 1.9 | 0.069 |
| 2.0 | 0.073 |

A particle size B illustrated in Table 2 represents a size of the second contrast substance which can be enlarged up to the particle sizes in which the “identification determination” shows the results of 1 to 3. If the second contrast substance is prepared so as to have the respective sizes of the particle size B in Table 2, the second contrast substance can be enlarged and displayed with a size of the corresponding particle size A on the image display monitor. In other words, as illustrated in Table 2, if the second contrast substance has a particle size of 0.007 mm or larger, the second contrast substance can be displayed on the image display monitor so that the above-described “identification determination” becomes two or more (i.e., becomes more clearly identified).

Table 3 illustrated below shows results of a visual inspection in which the second contrast substance is visually recognized based on the X-ray image obtained through the application example.

TABLE 3

| | | Brightness A of | Brightness A of |
|---|---|---|---|
| Brightness | Identification | Second Contrast | First Contrast |
| Difference | Determination | Substance | Substance |
| 0 | — | 30 | 30 |
| 5 | 1 | 25 | 30 |
| 10 | 1 | 20 | 30 |
| 15 | 2 | 15 | 30 |
| 20 | 2 | 10 | 30 |
| 25 | 2 | 5 | 30 |
| 30 | 3 | 0 | 30 |

The brightness A of the second contrast substance and the brightness A of the first contrast substance in Table 3 represent brightness of each contrast substance used in the application example. Here, the brightness means brightness of colors in which 100 represents a white color and zero represents a black color. A white color reproduced on the image display monitor is thus equal to a brightness of 100, and a black color reproduced on the same image display monitor equal to a brightness of zero. The intermediate brightness values are on a linear scale between these two boundaries (i.e., the linear brightness scale goes from 0 (white) to 100 (black), and the brightness amount linearly increases on the scale between these boundaries). For example, a brightness value of 30 is defined as brightness of the 30th from the brightness of zero toward the brightness of 100, when the brightness 100 to zero is divided into 100 stages. In the similar manner, the brightness of 50 is defined as the brightness of the 50th from the brightness of zero toward the brightness of 100. The “identification determination” in Table 3 shows results in which visibility of the second contrast substance is confirmed by observing the X-ray image illustrated in FIG. 9A. The second contrast substance is prepared so that the particle size is 0.2 mm in which the result of the identification determination of the particle size shows two. The results of the identification determination are as follows.

3: The second contrast substance can be clearly identified at a position of 0.7 m away from the X-ray image (normal use).

2: The second contrast substance can be identified at the position of 0.7 m away from the X-ray image (normal use).

1: The second contrast substance can be identified at a position of 0.3 m away from the X-ray image (i.e., the second contrast substance cannot be readily identified at a position of 0.7 m away from the X-ray image).

According to these results, when the particle size of the second contrast substance is 0.2 mm, if the brightness difference between the first contrast substance and the second contrast substance is at least five or more on the linear brightness scale described above, it is possible to obtain a result that the second contrast substance can be identified on the X-ray image. In view of the above-described results of the application example relating to the particle size, if the brightness difference is five or more on the linear brightness scale and the particle size of the second contrast substance is 0.2 mm or larger (when enlargedly displayed, 0.007 mm or larger), the particle size of the second contrast substance can be identified similarly to the above-described results.

Table 4 shows results in which the first contrast substance and the second contrast substance are displayed on a general-purpose image display monitor to test the identification of the second contrast substance, based on the brightness difference. The brightness A of each contrast substance is the same as the brightness confirmed in the above-described application example (however, excluding when the brightness difference is zero). The brightness B of each contrast substance is changed to a value in which the brightness of each contrast substance is shown on Table 4 so that the brightness difference is the same as the brightness difference in the above-described application example.

TABLE 4

| | | | | | Brightness of | Brightness |
|---|---|---|---|---|---|---|
| | | Brightness of | Brightness of | | Second | of First |
| Brightness | Identification | Second Contrast | First Contrast | Identification | Contrast | Contrast |
| Difference | Determination A | Substance A | Substance A | Determination B | Substance B | Substance B |
| 0 | — | 30 | 30 | — | 50 | 50 |
| 5 | 1 | 25 | 30 | 1 | 45 | 50 |
| 10 | 1 | 20 | 30 | 1 | 40 | 50 |
| 15 | 2 | 15 | 30 | 2 | 35 | 50 |
| 20 | 2 | 10 | 30 | 2 | 30 | 50 |
| 25 | 2 | 5 | 30 | 2 | 25 | 50 |
| 30 | 3 | 0 | 30 | 3 | 20 | 50 |
| 35 | — | — | — | 3 | 15 | 50 |
| 40 | — | — | — | 3 | 10 | 50 |
| 45 | — | — | — | 3 | 5 | 50 |
| 50 | — | — | — | 3 | 0 | 50 |

In a test, a point which includes the same brightness as the brightness of the second contrast substance shown in Table 4 and whose size is 0.2 mm is plotted on a black-colored figure which includes the same brightness as the brightness of the first contrast substance shown in Table 4. In this manner, a grayscale image which includes the same brightness difference as that of the filling material scheduled to be used is artificially created. Then, the created grayscale image is displayed on a display monitor, and the identification of the second contrast substance is determined by observing the image. As a result, when the bright difference is zero, it becomes difficult to identify the second contrast substance. However, if the brightness difference is five or more on the linear brightness scale, it is possible to visually identify the second contrast substance mixed in the first contrast substance regardless of the brightness of each contrast substance. The identification determination A represents a determination result of the confirmation test with regard to each contrast substance A, and the identification determination B represents a determination result of the confirmation test with regard to each contrast substance B.

As can be confirmed from the results of the application example illustrated in FIGS. 9A and 9B, it is possible to clearly identify the first contrast substance and the second contrast substance on the X-ray image. In addition, it is possible to confirm a flowing state of the whole filling material by observing the second contrast substance when the filling material is used. It is thus possible to recognize a state where the filling material is injected into the container, by confirming a flowing state of the whole filling material.

The filling material and the application example of the filling material have been described with reference to the embodiment and the parameters of the application example. However, the present invention can be appropriately changed within the scope defined in claims, and is not limited to the above-described embodiment and application example. For example, the filling material has been explained in reference to the implant indwelling practice to maintain a gap between adjacent spinous processes so as to be in a broadened state. However, the filling material is not limited to this intended use. The filling material can be widely applied in medical practices such as a space forming practice for forming a cavity inside a bone, a space broadening practice for temporarily broadening a space between living body organs, and additionally various manual skills (i.e., manually conducted operations) for confirming an injection state of a filling material when the filling material is injected into a container introduced into a living body.

Next, various embodiments relating to the implant indwelling practice for causing a flexible container to indwell between spinous processes will be described as an example. With regard to the same members as those in the above-described embodiment, description thereof will be omitted.

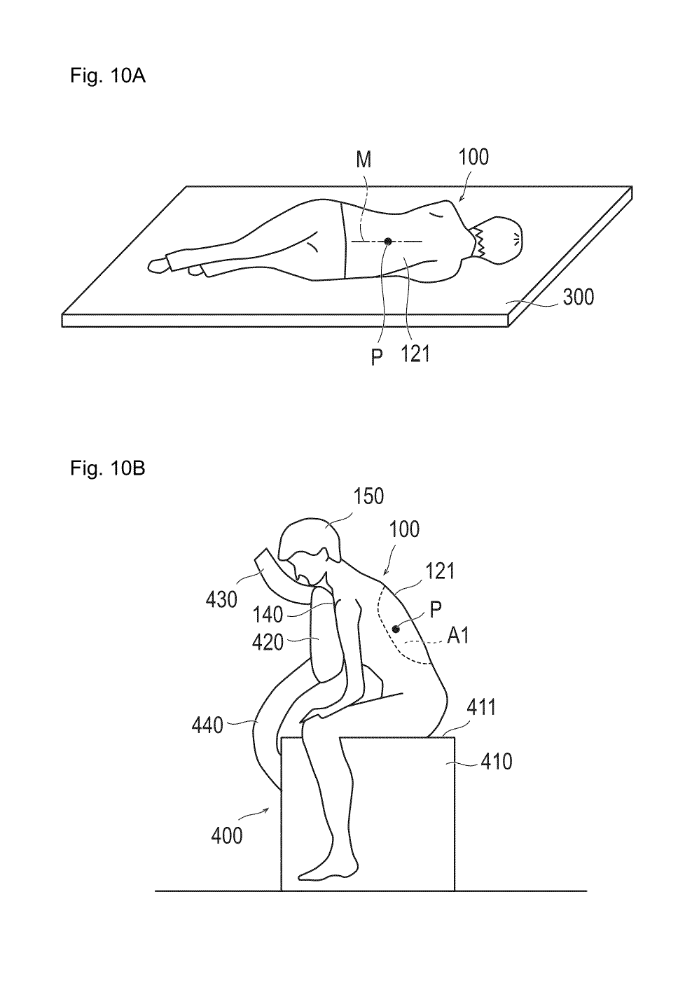

FIG. 10 illustrates a preferable posture which a subject can adopt when the medical device and filling material disclosed here is used.

When the flexible container 80 is introduced into the living body 120 of the subject 100, the subject 100 should take a position so that an operator can observe the back 121 of the subject 100. For example, it is possible to employ the laterally lying posture illustrated in FIG. 10A, the sitting posture illustrated in FIG. 10B, or alternatively a posture of lying face down and the like.

When the medical device and filling material disclosed here is used are used in a case of the laterally lying posture as illustrated in FIG. 10A, the subject 100 is laid on a mat 300,

On the other hand, when the medical device and filling material disclosed here is used in a case of the sitting posture as illustrated in FIG. 10B, it is possible to use a predetermined medical support device 400 as illustrated. The medical support device 400 can be configured to have a seat section 410 which has a seat surface 411 on which the subject 100 sits, a first support section 420 which can support the periphery of a chest 140 of the subject 100, a second support section 430 which can support a head (or a face) 150 of the subject 100 while being arranged above the first support section 420, and a holding section 440 which holds the first support section 420 so as to be swingable with respect to the seat section 410.

The first support section 420 and the second support section 430 are formed to have a curved outer shape so as not to apply a load to the subject 100 when being pressed against the chest 140 or the head 150 of the subject 100. In order to further minimize the load to the subject at the first support section 420 and the second support section 430, it is possible to install a member such as a cushion in these support sections 420 and 430.

The holding section 440 can be configured to elastically support the body weight of the subject 100 so that an excessively heavy load is not applied to the subject 100 when the body weight of the subject 100 is applied to the holding section 440.

When the subject 100 sits on the seat section 410, the subject 100 can lean the chest 140 against the first support section 420, and lean the head 150 against the second support section 430. In this manner, the subject 100 can lean his or her whole body against the medical support device 400. In addition, the subject 100 can adopt a posture in which the subject 100 put his or her two arms around the first support section 420. If the subject 100 adopts this posture, the subject 100 can lean his or her body on the medical support device 400 in a relaxed state.

When the medical support device 400 is used, the subject 100 can relatively freely change his or her posture while the medical device and filling material disclosed here is manually operated. Accordingly, the subject 100 is not forced to adopt an unfavorable posture over a long period of time, and the medical device and filling material disclosed here can be used while the subject 100 is in a relaxed state. In addition, an operator can sit on a chair or the like to use the medical device and filling material while confirming a practice area A1 of the subject 100. Accordingly, it is also possible to reduce the burden on the operator.

When the medical device and filling material disclosed here is used, it is preferable that the subject 100 is caused to adopt a posture in which the back 121 is bent rearward (i.e., rounded outwardly). If the medical device and filling material disclosed here is used in a state where the subject 100 adopts this posture, it becomes easy to confirm the practice area A1, and it becomes easy to guide the needle tip 25 of the puncture member 20 to a portion between the adjacent spinous processes 123a and 123b. Accordingly, it is possible to more quickly and safely use the medical device and filling material.

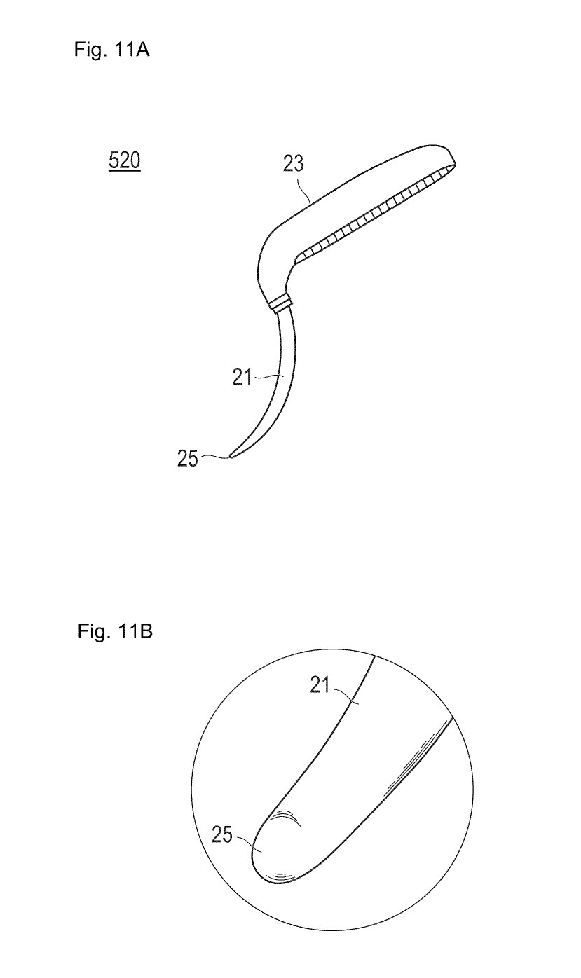

FIG. 11 illustrates Modification Example 1 of a puncture member.

The puncture member 20 illustrated in FIG. 2 is formed so that the needle tip 25 of the needle portion 21 has a sharply pointed shape. On the other hand, the needle tip 25 of a puncture member 520 illustrated in the present modification example is formed so as to have a rounded and curved shape as illustrated in FIG. 11B. According to the puncture member 520 in which the needle tip 25 is configured to have this rounded and curved shape, it is possible to minimize the risks of erroneous puncture such as puncture at a wrong position (bones, living body organs, or the like) and erroneous puncture in the body of the operator. However, when this puncture member 520 is used, it becomes difficult to smoothly pierce the living body 120. Accordingly, it is preferable to cut the skin of the living body 120 prior to the puncture. A method of cutting the skin varies depending on the size and shape of the container 80 introduced into the living body. For example, it may be preferable to cut the skin using the width of 2 mm to 20 mm and the depth of 2 mm to 20 mm, and more preferably the width of 5 mm to 10 mm and the depth of 10 mm to 15 mm.

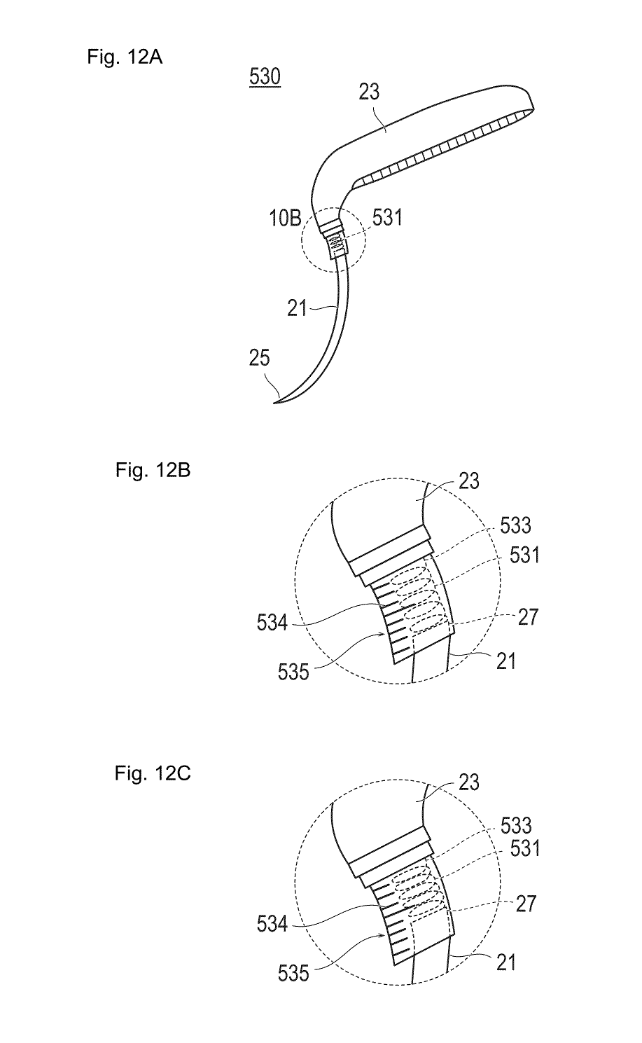

FIG. 12 illustrates Modification Example 2 of a puncture member.

A puncture member 530 illustrated in the present modification example is configured to measure puncture resistance received by the needle tip 25 of the needle portion 21 when the puncture member 530 is used.

As illustrated in FIGS. 12A and 12B, a spring 531 including a predetermined spring constant is attached to a proximal end 27 of the needle portion 21 of the puncture member 530. In addition, a proximal end 533 of the spring 531 is attached to the main body portion 23 of the puncture member 530. Therefore, the needle portion 21 is attached to the main body portion 23 via the spring 531.

A display portion 535 attached to a predetermined scale 534 is disposed in the main body portion 23. The display portion 535 is configured to include a transparent member having a cylindrical outer shape. Therefore, it is possible to visually see the proximal end 27 of the needle portion 21 arranged inside the display portion 535 from the outside.

The scale 534 is attached to the display portion 535 and is associated with a compression amount of the spring 531. The scale 534 thus allows the operator to visually confirm the puncture resistance (load) received by the needle tip 25 (i.e., depending on the position of the proximal end 27 as shown in FIG. 12B) when the spring 531 is compressed. For example, FIG. 12B illustrates when the puncture is performed and the spring 531 is not compressed, and FIG. 12C illustrates when the spring 531 is compressed and thus the proximal end 27 of the needle portion 21 is moved. Observing the scale 534 makes it is possible to confirm the puncture resistance received by the needle tip 25 of the needle portion 21. A form of causing the display portion 535 to display the puncture resistance may employ a configuration in addition to the configuration of using the illustrated scale. For example, a configuration may be adopted in which the puncture resistance is measured by using a potentiometer or the like so as to digitally display the measurement result.

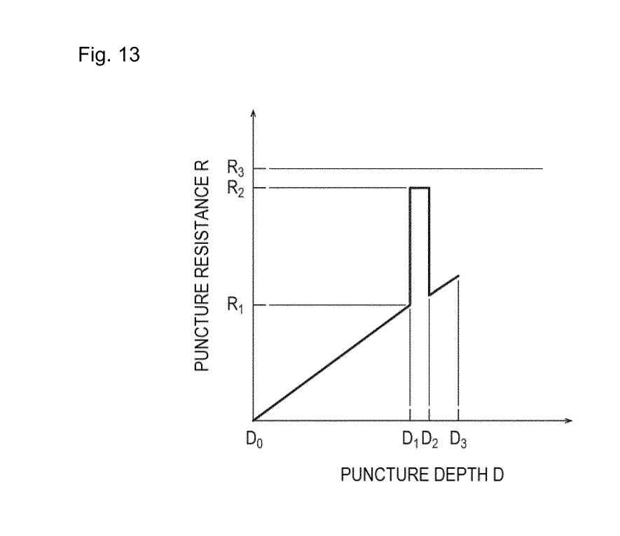

FIG. 13 briefly illustrates a general relationship between puncture depth D and puncture resistance R.

As illustrated in FIG. 13, if the needle portion 21 pierces the living body 120 through a puncture position D₀ (surface layer of the skin) and the needle tip 25 is moved forward into the living body 120, the puncture resistance gradually increases as the needle tip 25 further moves forward. In addition, if the needle tip 25 reaches an interspinous ligament present between the adjacent spinous processes 123a and 123b, the puncture resistance rapidly increases as illustrated (R₁ and R₂ in the drawing), in regions (D₁ and D₂ in the drawing) where the interspinous ligament is present. Then, after the needle tip 25 passes through the interspinous ligament, the puncture resistance rapidly decreases. Thereafter, the puncture resistance gradually increases in accordance with the puncture depth. In this manner, depending on the position where the needle tip 25 of the needle portion 20 of the puncture member 530 reaches, the puncture resistance applied to the needle tip 25 varies.

For example, when the puncture member 530 illustrated in FIGS. 12A to 12C is used, the puncture work is carried out while the puncture resistance is confirmed. This allows the operator to easily confirm any position at which the needle tip 25 of the needle portion 21 is moved forward. In addition, when calcification has occurred in the interspinous ligament, it is possible to confirm the progress of the calcification, based on the measured puncture resistance. In accordance with the progress of the calcification, the filling material injection pressure can be adjusted. In this manner, regardless of the presence or absence of the calcification, it is possible to smoothly broaden a gap between the adjacent spinous processes 123a and 123b. In addition, as illustrated in FIG. 13, when the measured puncture resistance indicates a predetermined value R₃, there is a possibility that the spinous process, a peripheral bone, or the like may be erroneously punctured. Since the puncture member is used, it is possible to quickly confirm this erroneous puncture. Accordingly, it is possible to manually operate in a less invasive manner.

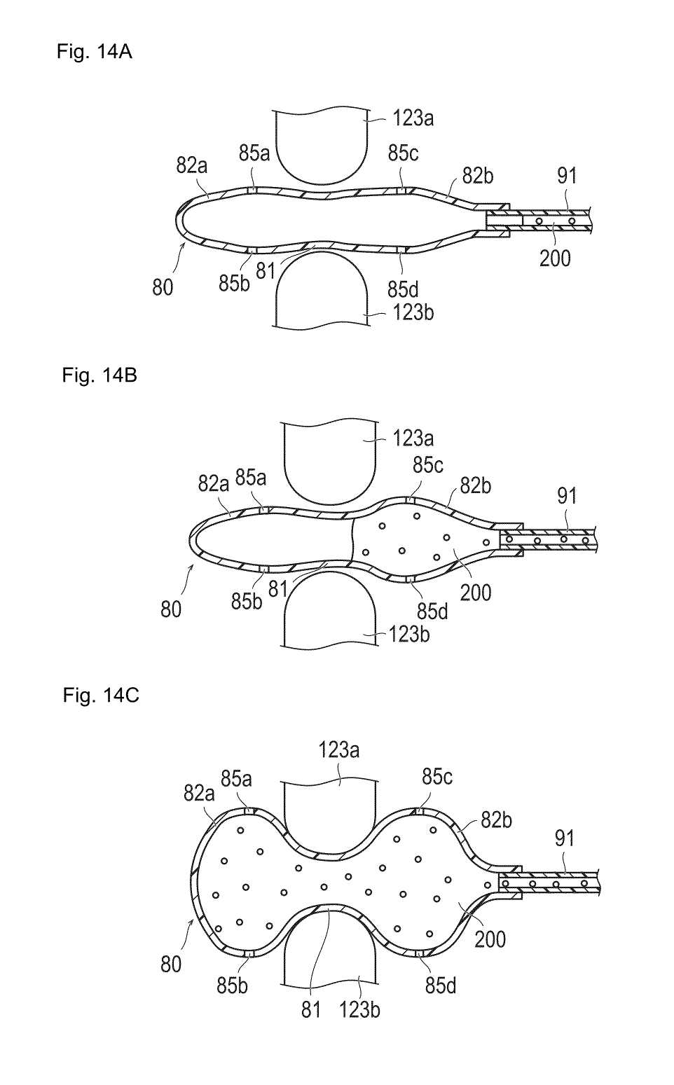

FIGS. 14A to 14C illustrate a modification example of a flexible container. As illustrated in FIG. 14A, X-ray contrast markers 85a to 85d can be attached to both end portions 82a and 82b of the flexible container 80. The respective X-ray contrast markers 85a to 85d can be configured to include a known material used in the medical field. For example, a configuration can be adopted in which platinum (Pt), Pt alloy, tungsten (W), W alloy, silver (Ag), Ag alloy, or the like is incorporated, or in which these materials are joined to the outer surface of the container 80. The size of each of the X-ray contrast markers 85a to 85d is not particularly limited. However, it is preferable to set the size of the X-ray contrast markers 85a to 85d so that the X-ray contrast markers 85a to 85d can be identified on a captured X-ray image.

As illustrated in FIG. 14A, when the work is carried out in order to arrange the flexible container 80 between the adjacent spinous processes 123a and 123b, it is possible to arrange the body portion 81 of the flexible container 80 between the adjacent spinous processes 123a and 123b through simple positioning, by confirming each position of the X-ray contrast markers 85a and 85b attached to the distal side and the X-ray contrast markers 85c and 85d attached to the proximal side.

As illustrated in FIGS. 14B and 14C, if the filling material 200 is injected into the flexible container 80 and the flexible container 80 expands, the respective X-ray contrast markers 85a to 85d are moved outward in the radial direction of both end portions 82a and 82b. At this time, it is possible to confirm that the flexible container 80 expands to a predetermined size by confirming whether the respective X-ray contrast markers 85a to 85d are moved to upper and lower positions in the drawing from the position of the space between the adjacent spinous processes 123a and 123b. Therefore, it is possible to easily confirm an expansion state of the flexible container 80 based on the X-ray image. Image capturing may be performed with an attached scale such as a ruler on the surface of the living body to measure a distance between the X-ray contrast markers 85a and 85b or a distance between the X-ray contrast markers 85c and 85d. In this manner, it is possible to measure outer dimensions of the flexible container 80 during the expansion.

In the example illustrated in FIG. 14, the X-ray contrast markers 85a to 85d are respectively attached to the upper end and the lower end of both end portions 82a and 82b. However, in order to recognize an expansion degree when the flexible container 80 expands, the X-ray contrast markers 85a to 85d may be attached to at least any one of the upper end side and the lower end side. The configuration can be appropriately changed. In addition, without attaching the X-ray contrast marker to both end portions 82a and 82b, a configuration can be adopted in which the X-ray contrast marker is attached to only one end portion. Even according to this configuration, the X-ray contrast marker functions as a guide to indicate the expansion degree of the flexible container 80 when the flexible container 80 expands.

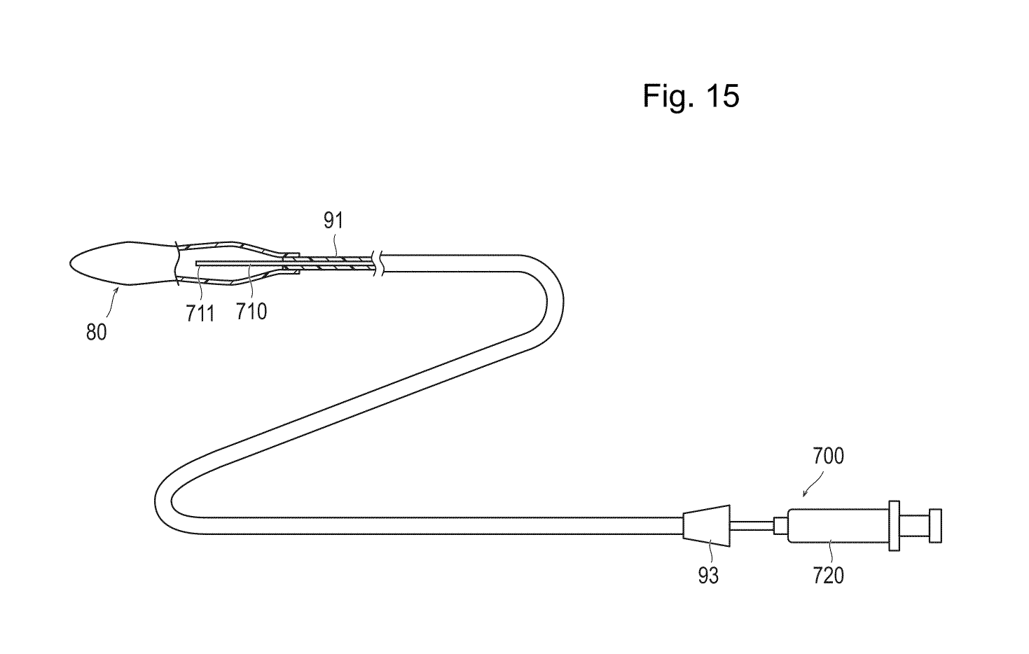

FIG. 15 illustrates a catheter system used for injecting the filling material into the flexible container.

A catheter system 700 illustrated in FIG. 15 has a thin catheter main body 710 which can be inserted into the above-described catheter tube 91 (refer to FIG. 4) and a filling material supply device 720 which is configured to be connectable to and detachable from the proximal end of the catheter main body 710.

A connector 93 into which the catheter main body 710 of the catheter system 700 can be inserted is disposed in the proximal end of the catheter tube 91. The connector 93 includes a function as a valve body for preventing the leakage of the filling material 200.

The catheter main body 710 of the catheter system 700 has the same configuration as that of a known catheter which is used in the medical field, and is a flexible body configured to include a resin material or the like. Although the illustration is omitted, an X-ray contrast marker is attached to a distal end 711 of the catheter main body 710 of the catheter system 700.

For example, as the filling material supply device 720, it is possible to use a syringe pump known in the medical field.

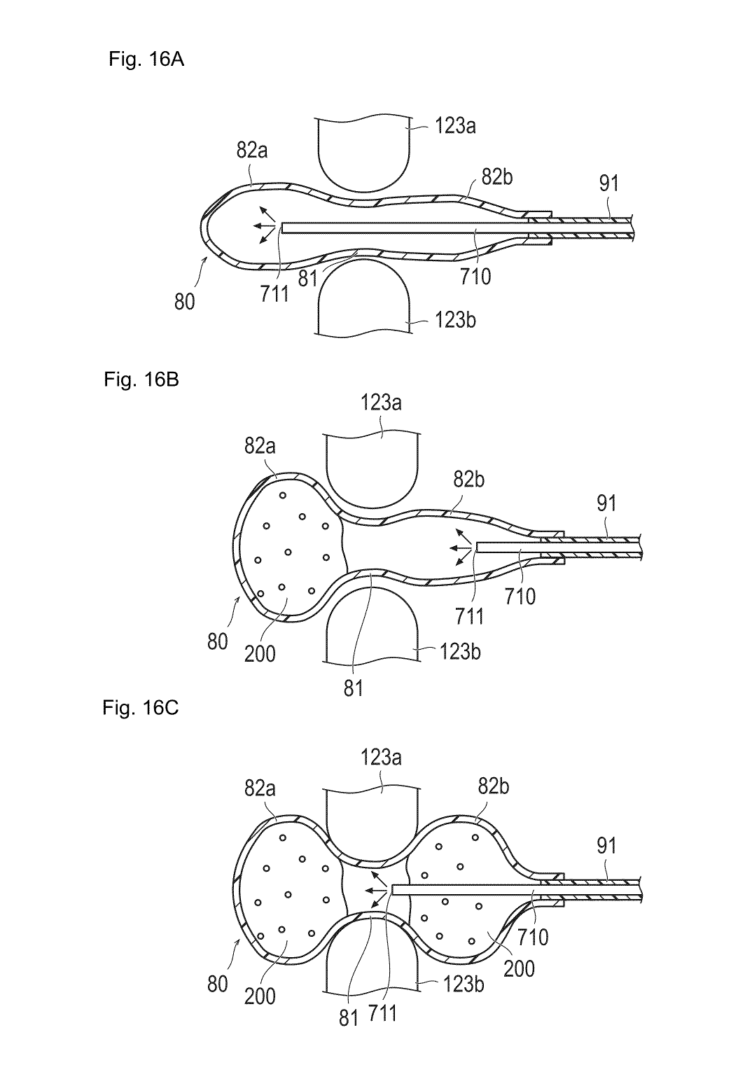

An example in which the catheter system 700 is used will be described with reference to FIGS. 16A to 16C.

As illustrated in FIG. 16A, the distal end 711 of the catheter main body 710 is moved to the distal side end portion 82a of the flexible container 80. A position of the distal end 711 of the catheter main body 710 can be confirmed by the X-ray contrast marker attached to the distal end 711. The filling material 200 is injected via the catheter main body 710 (the injection of the filling material is illustrated by an arrow in FIGS. 16A to 16C) so as to expand the distal side end portion 82a of the flexible container 80.

Next, as illustrated in FIG. 16B, the distal end 711 of the catheter main body 710 is moved to the proximal side end portion 82b of the flexible container 80 and the filling material 200 is injected into the proximal side end portion 82b of the flexible container 80.

Then, as illustrated in FIG. 16C, after the distal end 711 of the catheter main body 710 is moved to the body portion 81 of the flexible container 80 (i.e., an intermediate portion between the distal portion 82a and the proximal portion 82b), the filling material 200 is injected so as to expand the body portion 81.

As described above, both end portions 82a and 82b of the flexible container 80 are selectively expanded earlier than the body portion 81 of the flexible container 80. This order of filling material 200 injection makes it possible to prevent a slipping phenomenon from occurring when the flexible container 80 is expanded. The slipping phenomenon is when the body portion 81 is expanded earlier than both end portions 82a and 82b of the flexible container 80, thereby pressing the body portion 81 against the respective spinous processes 123a and 123b. Consequently, a force acting in a direction away from the respective spinous processes 123a and 123b is applied to the flexible container 80, thereby causing misalignment of the flexible container 80.

The catheter system 700 is used to expand both end portions 82a and 82b earlier than the body portion 81. In this manner, the body portion 81 can be expanded in a state where the flexible container 80 is aligned with the respective spinous processes 123a and 123b by using both end portions 82a and 82b. According to this configuration, it is possible to prevent the slipping phenomenon from occurring as described above. In order to prevent the occurrence of the slipping phenomenon, the expansion may start from any one of both end portions 82a and 82b of the flexible container 80. For example, after the proximal side end portion 82b is expanded, the distal side end portion 82a may be expanded. In this order, the work for injecting the filling material 200 can be carried out.

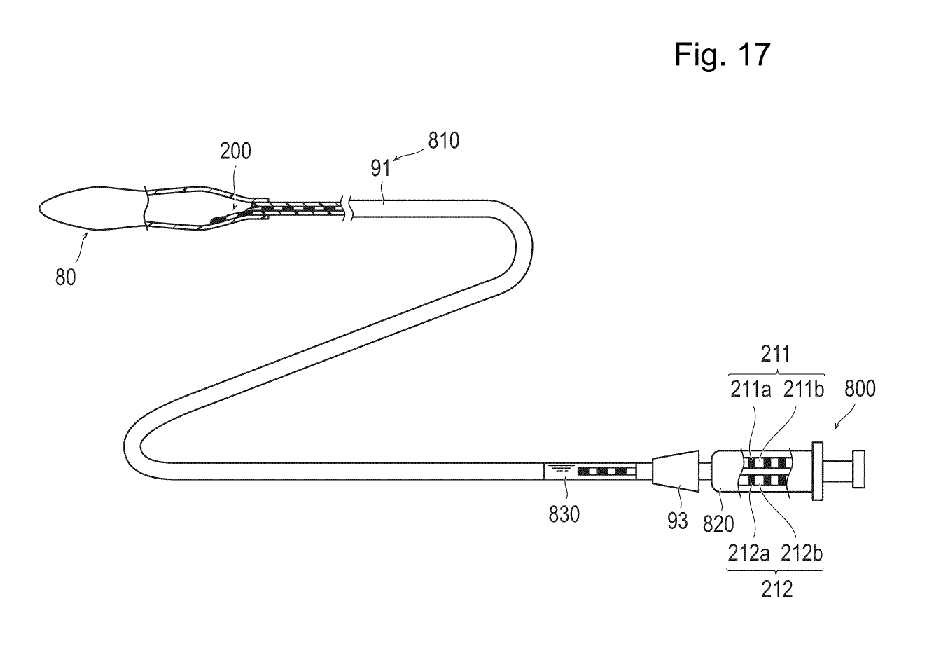

FIG. 17 illustrates a modification example of a catheter system used for injecting the filling material into the flexible container.

A catheter system 800 illustrated in the present modification example is configured to allow an operator to easily confirm the state of the filling material 200 injection into the flexible container 80.

A catheter main body 810 included in the catheter system 800 is configured to be connectable to and detachable from the flexible container 80. That is, according to the present modification example, the catheter tube 91 used for injecting the filling material 200 into the flexible container 80 is configured to include the catheter main body 810 of the catheter system 800. A filling material supply unit 820 is interlocked with the catheter main body 810 via the connector 93 disposed in the catheter main body 810 in a liquid-tight and an air-tight manner.

The filling material supply unit 820 included in the catheter system 800 is configured to be capable of holding two types of fluid constituting the filling material 200. For example, it is possible to use the types of fluid which are described in the previous embodiment. The present embodiment employs a filling material which causes a curing reaction by mixing a first fluid 211 such as the two-liquid mixture cross-linked polymer and a second fluid 212 with each other.

The respective fluids 211 and 212 are prepared in a state where first portions 211a and 212a and second portions 211b and 212b with different X-ray contrast properties are alternately stacked on each other. The respective fluids 211 and 212 are held in the filling material supply unit 820 so that the first portions 211a and 212a with higher contrast X-ray contrast properties are simultaneously injected into the flexible container 80, and so that the second portions 211b and 212b with respectively lower contrast X-ray contrast properties are simultaneously injected into the flexible container 80.

If the filling material 200 is injected using the catheter system 800, the portions 211a and 212a whose X-ray contrast properties are higher and the portions 211b and 212b whose X-ray contrast properties are lower are alternately injected. Therefore, it is possible to observe a state where the portions whose X-ray contrast properties are higher and the portions whose X-ray contrast properties are lower are injected so as to be alternately stacked on each other on the X-ray image. It is thus possible to recognize that the inside of the flexible container 80 is filled with the filling material 200 by confirming that there is no movement of the portions with higher contrast X-ray properties. Accordingly, it is possible to complete the work for injecting the filling material 200 at the preferable timing.

For example, a transparent window portion 830 which enables a user to observe the inside of the catheter main body 810 from the outside can be disposed in the catheter main body 810 of the catheter system 800. It is possible to confirm the timing for injecting the filling material 200 by observing through the window portion 830.

The window portion 830 can be created by manufacturing a portion of the catheter main body 810 out of a transparent resin material or by configuring the catheter main body 810 to partially include transparent glass. A position, a size, or the like of the window portion 830 is not particularly limited as long as a flow of the filling material 200 can be confirmed. The window portion 830 can also be disposed in the above-described catheter tube 91 in each embodiment, or in the catheter main body 710 of the catheter system 700.

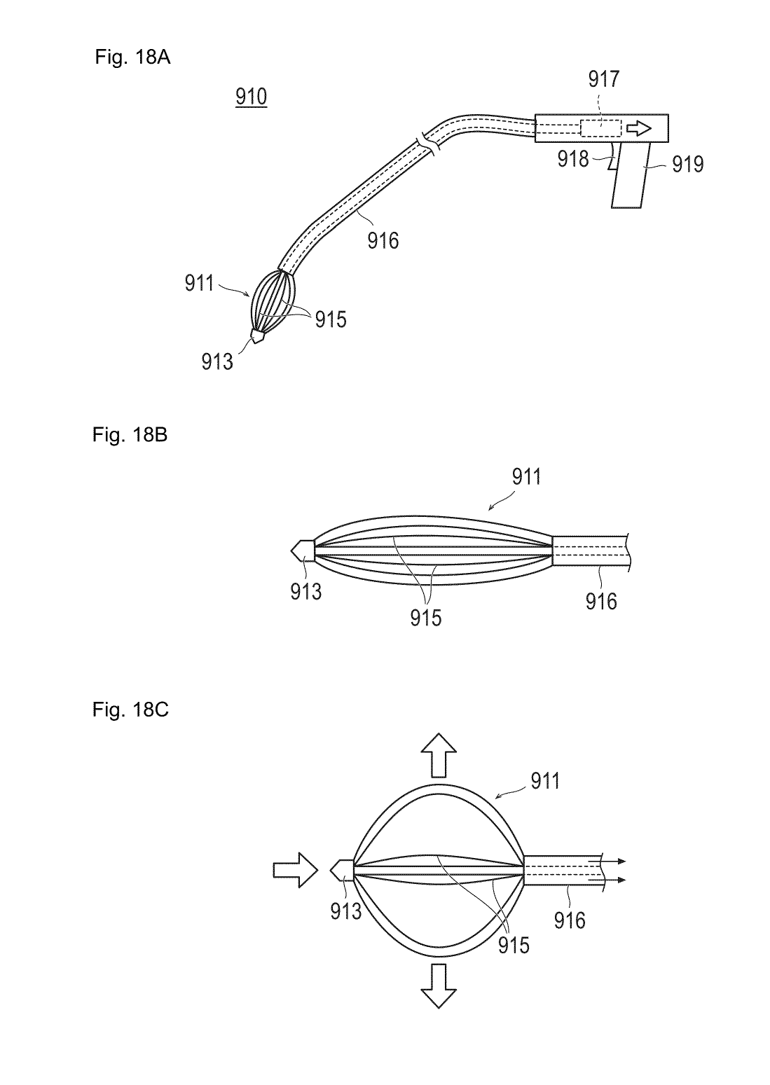

FIGS. 18A to 18C illustrate an assisting device used in order to broaden a gap between the adjacent spinous processes.

When manual skills are used in order to cause the flexible container 80 having the injected filling material 200 to indwell between the adjacent spinous processes 123a and 123b, for example, it is possible to use manual skills for preliminarily broadening a gap between the adjacent spinous processes 123a and 123b (manual skills for preliminary broadening).

Depending on the subject 100, the interspinous ligament of the subject 100 may be previously calcified. If the calcification has occurred, the interspinous ligament cannot be spread out only by using expansion pressure of the flexible container 80 having the injected filling material 200. Consequently, there is a possibility that the flexible container 80 cannot expand to a sufficient size. Therefore, prior to the expansion of the flexible container 80, a cavity is formed inside the interspinous ligament. In this manner, the manual skills for preliminary broadening are used which enable the flexible container 80 to smoothly expand between the adjacent spinous processes 123a and 123b. An assisting device 910 illustrated in FIG. 18 can be used for the manual skills for preliminary broadening.

A configuration of each unit of the assisting device 910 will be described with reference to FIGS. 18A to 18C.

The assisting device 910 has an expansion member 911 which is formed of multiple leaf springs arranged in the distal end, a distal end stopper 913 attached to the distal end of the expansion member 911, a wire 915 interlocked with the distal end stopper 913, a flexible shaft unit 916 into which the wire 915 is inserted, a slide member 917 interlocked with the wire 915, and a hand operation unit 919 which includes a trigger 918 for operating the movement of the slide member 917.

The expansion member 911 is configured to include four leaf springs which are arranged in the circumferential direction so that the outer shape of the expansion member 911 becomes substantially elliptical (i.e., the expansion member 911 may expand into a three-dimensional elliptical shape). The number, a shape, or the like of the leaf spring is not particularly limited as long as the expansion member 911 is configured to be expandable.

Examples of material for the wire 916 include metal, a resin, or the like which is machined into a thin string shape. In the illustrated example, two wires 915 are used, but the number of wires 915 is not particularly limited.

For example, the shaft unit 916 can be configured to include the same material as that of the catheter tube 91 in the above-described embodiment. In addition, the shaft portion 916 includes the same flexibility as that of the catheter tube 91.

The distal end of the wire 915 is fixed to the distal end stopper 913 by using a known method such as fusing, welding, an adhesive, and the like. In addition, the distal end stopper 913 is also fixed to the expansion member 911 by using the above-described fixing method.

The slide member 917 arranged inside the hand operation unit 919 is configured to move rearward (i.e., proximally) in a direction of an arrow in the drawing when the trigger 918 is pressed, and to move in a direction opposite to the arrow (i.e., distally) when pressing of the trigger 918 is released.

FIG. 18B illustrates the expansion member 911 in a state before expansion. If the trigger 918 is pressed in this state, the slide member 917 moves rearward (i.e., proximally), and the distal end stopper 913 correspondingly moves rearward (i.e., proximally) after being drawn by the wire 915 (i.e., the wire 915 applies a force in the proximal direction to the distal end stopper 915). As a result, as illustrated in FIG. 18C, the expansion member 911 is expanded and deformed after being pressed by the distal end stopper 913. The expansion member 911 is expanded and deformed, thereby pressing the interspinous ligament present around the expansion member 911. In this manner, it is possible to form a predetermined cavity between the adjacent spinous processes 123a and 123b. If the trigger 918 is released, an elastic restoring force of the leaf spring causes the expansion member 911 to restore its original shape.

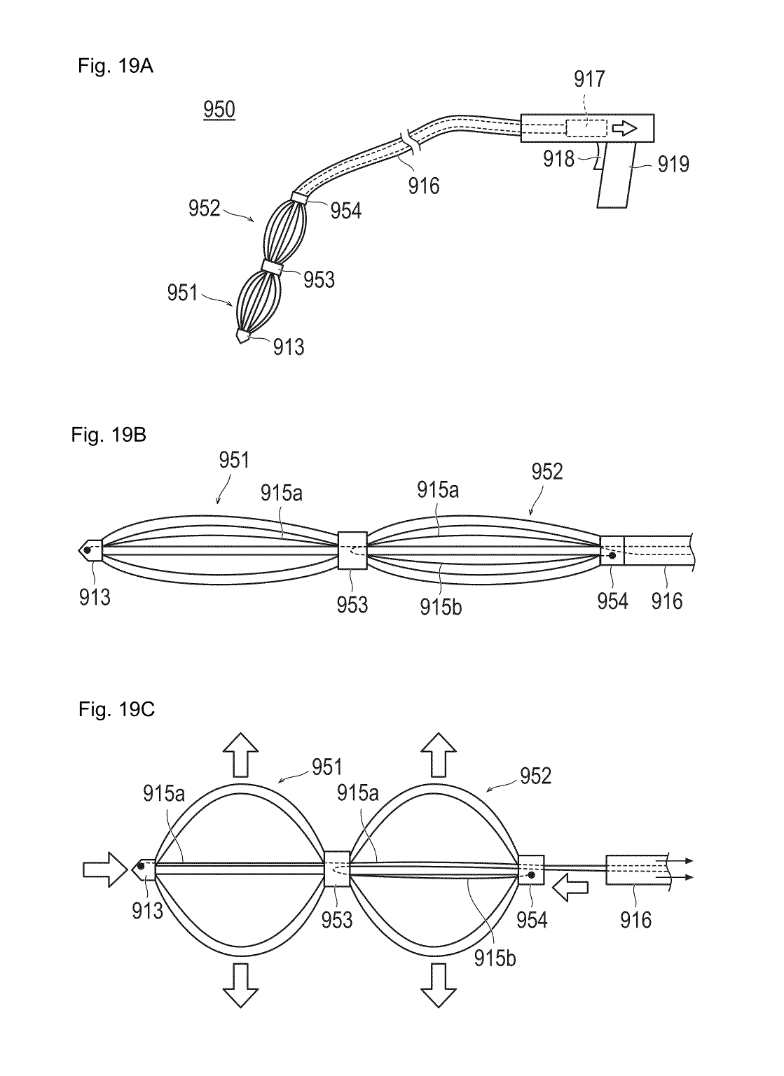

FIGS. 19A to 19C illustrate a modification example of an assisting device used in order to broaden a gap between the adjacent spinous processes.

An assisting device 950 according to the present modification example has a first expansion portion 951 which is arranged on the distal side and a second expansion portion 952 which is arranged on the proximal side. In this regard, the assisting device 950 is different from the above-described assisting device 900 (i.e., because there are two expansion portions—a distal expansion portion 951 and a proximal expansion portion 952).

According to this assisting device 950, a shape of a cavity formed by the assisting device 950 can be formed so as to have substantially the same shape as the shape of the flexible container 80 (e.g., as in FIG. 6) in the above-described embodiment, when the flexible container 80 expands. Accordingly, the flexible container 80 can be more smoothly expanded.

A configuration of each unit of the assisting device 950 will be described with reference to FIGS. 19A to 19C.

The first expansion portion 951 and the second expansion portion 952 are configured to simultaneously expand in response to a pressing operation of the trigger 918. In order to enable this operation, an intermediate stopper 953 is arranged between the first expansion portion 951 and the second expansion portion 952, and a proximal end stopper 954 is arranged in the distal end of a shaft member 916.

The assisting device 950 includes two wires. The first wire 915a has its distal end fixed to the distal end stopper 913, and the second wire 915b is inserted into the intermediate stopper 953 so as to be folded back toward the proximal side inside the intermediate stopper 953. The distal end of the second wire 915b is fixed to the proximal end stopper 954. The first wire 915a and the second wire 915b are inserted into the proximal end stopper 954, and the proximal end stopper 954 is locked with the respective wires 915a and 915b. In addition, the proximal end of the respective wires 915a and 915b is fixed to the slide member 917. If the slide member 917 moves forward and rearward (i.e., distally and proximally), the respective wires 915a and 915b are configured to move forward (i.e., distally) or to move rearward (i.e., proximally) in line with the forward and rearward movements of the slide member 917. A black circle in the drawing illustrates a fixing point between each stopper and the wire.

As illustrated in FIG. 19C, if the trigger 918 is pressed, the distal end stopper 913 moves rearward (i.e., proximally), and the proximal end stopper 954 moves forward (i.e., distally). At this time, the intermediate stopper 953 does not move, and stays at the same position as that before and after the trigger 918 is pressed. Therefore, the distal end stopper 913 moves toward the intermediate stopper 953. In this manner, the first expansion portion 951 interposed between the distal end stopper 913 and the intermediate stopper 953 is pressed, compressed, and expanded in the circumferential direction. In addition, the proximal end stopper 954 moves toward the intermediate stopper 953. In this manner, the second expansion portion 952 interposed between the proximal end stopper 954 and the intermediate stopper 953 is pressed, compressed, and expanded in the circumferential direction. As described above, the first expansion portion 951 and the second expansion portion 952 can be simultaneously expanded by operating the trigger 918.

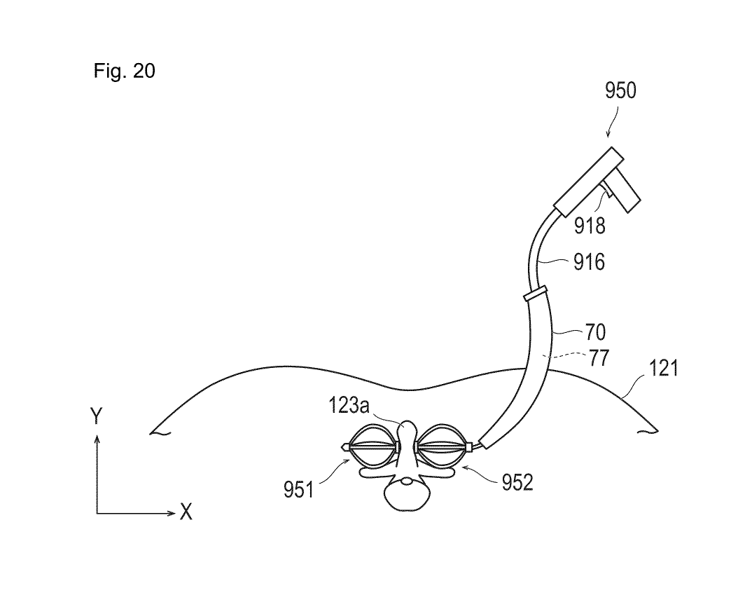

FIG. 20 illustrates an example in which the assisting device 950 is used.

The assisting device 950 can be introduced into the living body 120 of the subject 100 by jointly using the guide member 70 which is used in order to introduce the flexible container 80 into the living body 120.

As described above, the shaft member 916 of the assisting device 950 is configured to be flexible. The assisting device 950 is pushed into the lumen 77 of the guide member 70 from the distal side while the expansion portions 951 and 952 of the assisting device 950 are not yet expanded (i.e., are in a non-expanded or collapsed state). In this manner, the respective expansion portions 951 and 952 can be arranged around the adjacent spinous processes 123a and 123b as illustrated.

After the respective expansion portions 951 and 952 are arranged at a predetermined position as illustrated, the trigger 918 is pressed (i.e., operated), thereby forming a cavity between the adjacent spinous processes 123a and 123b. Thereafter, the respective expansion portions 951 and 952 are contracted. The assisting device 950 is removed out from the living body 120 via the lumen 77 of the guide member 70. Thereafter, the flexible container 80 is introduced into a portion between the adjacent spinous processes 123a and 123b via the lumen 77 of the guide member 70, and then the filling material 200 is injected into the container 80. After the preliminary broadening is performed, the flexible container 80 is expanded. Accordingly, the container 80 can be smoothly expanded and deformed. In addition, in this case, the puncture member 20 is re-assembled to the guide member 70, and the needle tip 25 of the needle portion 21 of the puncture member 20 is caused to reach the indwelling target position P. In this manner, it is possible to carry out work for pressing the guide member 70 so as to move forward in the distal end direction.

In addition, for example, the indwelling work may be performed in the following order. The shaft member 916 of the assisting device 950 is formed to be sufficiently thin and the flexible container 80 is inserted into the living body 120. Thereafter, the assisting device 950 is removed after the respective expansion portions 951 and 952 expand inside the container 80. Thereafter, the filling material 200 is injected into the flexible container 80 within the living body.

In addition, the assisting device 910 and the assisting device 950 can be consecutively used by using a similar manual operation. It is possible to more reliably form a predetermined cavity between the adjacent spinous processes 123a and 123b by using the assisting device 910 and the assisting device 950.

The detailed description above describes embodiments of a filling material and a method of injecting the filling material representing examples of the filling material and the method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A filling material configured to be injected into an expandable and flexible container positioned within a living body, the flexible container being insertable into and removable from the living body, the filling material comprising:
a first contrast substance that is flowable and possesses X-ray contrast properties; and
a second contrast substance mixed with the first contrast substance, the second contrast substance being insoluble in the first contrast substance and possessing higher contrast X-ray contrast properties than the X-ray contrast properties of the first contrast substance.

2. The filling material according to claim 1, wherein a brightness difference between the second contrast substance and the first contrast substance is five or more when viewed on an X-ray image, the brightness difference being measured on a linear brightness scale in which a brightness value of white is 0 and the brightness value of black is 100.

3. The filling material according to claim 1, wherein the second contrast substance is scattered particles in the first contrast substance.

4. The filling material according to claim 3, wherein a particle size of the second contrast substance is equal to or larger than 0.007 mm, and is smaller than 2.0 mm.

5. The filling material according to claim 1, wherein the first contrast substance cures with a lapse of time.

6. The filling material according to claim 1, wherein the second contrast substance is a metal material.

7. The filling material according to claim 1,
wherein the flexible container is a medical device configured to indwell between adjacent spinous processes inside the living body, the flexible container maintaining a gap between the adjacent spinous processes when the filling material is injected into the flexible container, and
wherein the filling material expands the container to maintain an expansion state of the container.

8. A method of confirming an injection state of a filling material comprising:
injecting the filling material into a flexible container in a living body, the filling material including a first contrast substance that is flowable and possesses first X-ray contrast properties and a second contrast substance that is insoluble in the first contrast substance and possesses X-ray contrast properties higher in contrast than the X-ray contrast properties of the first contrast substance, the filling material being a mixture of the first contrast substance and the second contrast substance;
expanding the flexible container in the living body by increasing an amount of the filling material in the flexible container;
observing the second contrast substance on an X-ray image; and
confirming when a rate of change of the amount of the filling material is at a target rate of change based on the observation of the second contrast substance on the X-ray image.

9. The method according to claim 8, further comprising positioning the flexible container between adjacent spinous processes inside the living body before any of the filling material is injected into the flexible container.

10. The method according to claim 9, wherein
the flexible container possesses a distal portion, an intermediate portion, and a proximal portion; and
the injecting of the filling material causes the distal portion and the proximal portion of the flexible container to expand further than the intermediate portion of the flexible container.

11. The method according to claim 10, wherein the filling material injection comprises injecting the filling material in the distal portion of the flexible container, injecting the filling material in the proximal portion of the flexible container, and injecting the filling material is in the intermediate portion of the flexible container; and
the filling material is only injected into the intermediate portion of the flexible container after the filling material has been injected into the distal portion and the proximal portion of the flexible container.

12. The method according to claim 8, wherein
the filling material is injected into the flexible container via a catheter tube, the catheter tube configured to connect to the flexible container; and
the method further comprising removing the catheter tube from the living body after the rate of change of the filling material is confirmed at the target rate of change.

13. A combination comprising:
a flexible container configured to be inserted into a living body, the flexible container possessing an expandable proximal end;
a catheter tube connected to the flexible container; and
a filling material configured to be injected into the flexible container through the catheter tube to expand the flexible container while the flexible container is positioned in the living;
the filling material body comprising a mixture of a first contrast substance and a second contrast substance, the first contrast substance being flowable and possessing X-ray contrast properties, the second contrast substance being insoluble in the first contrast substance and possessing higher contrast X-ray contrast properties than the X-ray contrast properties of the first contrast substance.

14. The combination according to claim 13,
wherein a brightness difference between the second contrast substance and the first contrast substance is five or more on an X-ray image, the brightness difference being measured on a linear brightness scale in which the brightness value of white is 0 and the brightness value of black is 100.

15. The filling material according to claim 13, wherein the second contrast substance is scattered particles in the first contrast substance.

16. The combination according to claim 13, wherein a particle size of the second contrast substance is equal to or larger than 0.007 mm, and is smaller than 2.0 mm.

17. The combination according to claim 13, wherein the first contrast substance cures with a lapse of time.

18. The combination according to claim 13, wherein the second contrast substance is a metal material.

19. The combination according to claim 13,
wherein the flexible container is a medical device configured to indwell between adjacent spinous processes inside the living body, the flexible container maintaining a gap between the adjacent spinous processes when the filling material is injected into the flexible container, and
wherein the filling material expands the container to maintain an expansion state of the container.
